(19) United States
(12) Patent Application Publication
(10) Pub. No.: US 2021/0355441 A1
(43) Pub. Date: Nov. 18, 2021

(54) GENERATION OF HOXA-EXPRESSING HEMOGENIC ENDOTHELIUM WITH ENHANCED T CELL POTENTIAL FROM HPSCS

(71) Applicant: Wisconsin Alumni Research Foundation, Madision, WI (US)

(72) Inventors: Igor Slukvin, Verona, WI (US); Ho Sun Jung, Madison, WI (US)

(21) Appl. No.: 17/323,715

(22) Filed: May 18, 2021

Related U.S. Application Data
(60) Provisional application No. 63/026,494, filed on May 18, 2020.

(51) Int. Cl. C12N 5/078; C12N 15/63

(52) U.S. Cl. CPC C12N 5/0634; C12N 15/63; C12N 2501/26; C12N 2501/2307; C12N 2501/2306; C12N 2501/2303; C12N 2501/125

(57) ABSTRACT

The present invention provides methods of creating a population of hemogenic endothelial cells with arterial specification and enhanced T cell potential. The methods involve inducing the expression of a SOX17 transgene in human pluripotent stem cells starting at day 2 of differentiation. Stem cells that express the SOX17 transgene are also provided.

### CROSS-REFERENCE TO RELATED APPLICATIONS

This application claims priority to U.S. Provisional Application No. 63/026,494 filed on May 18, 2020, the contents of which are incorporated by reference in their entireties.

### STATEMENT REGARDING FEDERALLY SPONSORED RESEARCH

This invention was made with government support under HL142665, OD011106, CA014520, and HL134655 awarded by the National Institutes of Health. The government has certain rights in the invention.

### SEQUENCE LISTING

A Sequence Listing accompanies this application and is submitted as an ASCII text file of the sequence listing named “960296_04147_ST25.txt” which is 18.5 KB in size and was created on Apr. 30, 2021. The sequence listing is electronically submitted via EFS-Web with the application and is incorporated herein by reference in its entirety.

### BACKGROUND

De novo production of hematopoietic stem cells (HSCs) from in vitro expandable human cells, such as pluripotent stem cells (hPSCs), represents a promising approach for stem cell-based therapies and modeling of hematologic diseases. However, generation of HSCs and lymphoid cells from hPSCs remains a significant challenge. During development, blood cells and HSCs arise from hemogenic endothelium (HE) via a definitive hematopoiesis program that produces the entire spectrum of adult-type erythro-myeloid progenitors (EMP) and lymphoid cells. Importantly, these in vivo-produced cells have the capacity to provide long-term cell repopulation in an adult recipient following engraftment. Although previous studies have successfully generated hematopoietic progenitors (HPs) with a HSC phenotype and limited engraftment potential from pluripotent stem cells (PSCs), cells with the capacity for robust and consistent engraftment with recapitulation of the full spectrum of terminally differentiated hematopoietic cells, including lymphoid cells, has not been achieved. Thus, identifying key cellular and molecular programs required for proper lymphoid and HSC specification in vitro is essential to overcome the current roadblocks to advance the lymphoid cell and HSC manufacturing technology.

### SUMMARY

The present invention provides methods of enhancing HOXA gene expression and arterial specification of hemogenic endothelium with superior lymphomyeloid potential in differentiating human pluripotent stem cells (hPSCs). The methods comprise (a) introducing an inducible SOX17 transgene into a population of hPSCs; (b) culturing the hPSCs for at least two days under conditions to differentiate the hPSC into KDR⁺ mesoderm cells; and (c) inducing expression of the SOX17 transgene in the KDR⁺mesoderm cells and culturing for at least two days, such that DLL4⁺CXCR4⁺ arterial hemogenic endothelium (AHE) cells are obtained.

In some embodiments, the methods involve transducing the hPSCs with a vector comprising an inducible promoter operably linked to the SOX17 transgene.

In another aspect, the present invention provides cell populations produced by the methods disclosed herein.

In another aspect, the present invention provides isolated in vitro populations of DLL4⁺CXCR4⁺ AHE cells differentiated from an hPSC population comprising a SOX17 transgene.

In another aspect, the present invention provide a method of expansion of hematopoietic progenitors comprising a) generating hemogenic endothelium (HE) cells in presence of SOX17 upregulation; b) culturing the HE cells on OP9 or OP9-DLL4 cells in medium comprising FLT3L, TPO SCF, IL6, and IL3 for at least an additional 5 days; c) collecting the floating hematopoietic progenitor cells (HP); and d) culturing the HPs of step c in medium comprising FLT3L, TPO SCF, IL6, and IL3 for at least an additional 5 days to expand HPs with myeloid and lymphoid potential. In some aspects, the method further comprises (e) passaging the cells of step (d) for at least two weeks in medium comprising SCF, FLT3L and IL-7 for at least two weeks to produce CD4+CD8+ T cells.

In some embodiments, the cells are further differentiated to form an isolated in vitro T cell population that may be used for several downstream applications, such as the generation of exogenous chimeric antigen receptors (CARs).

### BRIEF DESCRIPTION OF THE DRAWINGS

FIG. 1 demonstrates that SOX17 knockout impairs arterial specification and definitive hematopoiesis. (A) Schematic diagram of hematopoietic development and in defined conditions. D is day of differentiation. (B) HB-CFC potential of wild-type or SOX17^{-/-} H19 cells (mean±SD, for 2 independent experiments performed in duplicates). **p<0.01, t-test. (C) Flow cytometric analysis of day 4 HE. (D) and (E) flow cytometric analysis of day 5 HE. Graphs show the percentages and total number of cells generated from 10⁴ hESCs (mean±SD, n=3 experiments). **p<0.01 and ***p<0.001, t-test. (F) Schematic diagram of experiments. Wild and SOX17^{-/-} cells were purified using CD31 MACS on day 4 and plated on OP9 or OP9-DLL4 for 5 days. (G) Hematopoietic colony-forming potential of day 4 HE after 5 days of culture on OP9 or OP9-DLL4 (mean±SD, n=2 experiments). ****p<0.0001, two-way ANOVA, Tukey's multiple comparisons test. (H) Flow cytometric analysis of T cell differentiation. (I) Graphs show the total number of T cells generated from 10⁴ CD43⁺ cells (mean±SD, n=3 experiments). ****p<0.0001, two-way ANOVA Tukey's multiple comparisons test. See also FIG. 8.

FIG. 2 demonstrates that SOX17 enhances arterial specification and definitive lympho-myeloid potential of HE at day 4 of differentiation. (A) Schematic diagram of experiments. (B) Effect of SOX17 overexpression during D2-3 on HB-CFCs (mean±SD, n=2 experiments). Graph shows HB-CFCs per 10⁴ cells collected on day 3 of differentiation. (C) Expression of arterial markers and Venus-reporter in iSOX17 cells on day 4 differentiation with or without DOX. Scale bars are 200 μm. (D) and (E) SOX17 overexpression increases the percentages and total numbers of VEC⁺ cells and AHE on day 4 of differentiation from 10⁴ hESCs. Results are mean±SD, n=3 experiments; **p<0.01, and ****p<0.0001, two-way ANOVA Sidak's multiple comparisons test. (F) CFC potential of HP collected following culture of day 4 HE on OP9 or OP9-DLL4 for 5 days (mean±SD, n=2 experiments performed in duplicates). *p<0.05, **p<0.01, ****p<0.0001, two-way ANOVA, Tukey's multiple comparisons test. (G) Graphs show the total number of T cells from produced from 10⁴ HPs collected following culture of D4 HE on OP9 or OP9-DLL4 for 5 days (mean±SD, n=3 experiments). *p<0.05, ***p<0.001, ****p<0.0001, two-way ANOVA Sidak's multiple comparisons test. See also FIG. 9.

FIG. 3 demonstrates that SOX17 induction promotes specification of DLL4⁺CXCR4⁺ AHE with superior lympho-myeloid potential on day 5 of differentiation. (A) Schematic diagram of experiments. (B) Flow cytometric analysis of HE on day 5 of differentiation in DOX+ and DOX− conditions. (C) DOX effect on HE formation on day 5 of differentiation (D5 HE). (D) DOX treatment enhances specification of DLL4⁺CXCR4⁺ arterial type HE. (C and D) Graphs show the percentages and total number of cells generated from 10⁴ hESCs (mean±SD and triplicate independent experiments). *p<0.05 and **p<0.01, ***p<0.001 two-way ANOVA, Sidak's multiple comparisons test. (E) Limiting dilution assay to determine the frequency of hemogenic progenitors in DLL4⁺CXCR4⁻ and DLL4⁺CXCR4⁺ HE cultures with or without DOX. (F) CFC potential of HPs collected after 5 day of culture of indicated day 5 HE subset (mean±SD, n=2 experiments performed in duplicates). **p<0.01, ***p<0.001, and **** p<0.0001 two-way ANOVA, Tukey's multiple comparisons test. (G) Graphs show the total number of T cells produced from 10⁴ HPs collected after 5 day of culture of indicated day 5 HE subset (mean±SD, n=2-3 experiments). *p<0.05 and **p<0.01, one-way ANOVA Tukey's multiple comparisons test. See also FIG. 10.

FIG. 4 shows molecular profiling of day 4 HE generated with and without DOX. (A) Expression fold changes between DOX+ and DOX− conditions for genes in selected KEGG pathways. GENCODE genes that have symbols mapping to the same KEGG pathway genes were removed. (B) Heat map represents expression of selected genes. (C) GO terms enriched for the top 5% of genes with ATAC-seq signals increased at promoters upon DOX induction. Selected enriched terms were required to have adjusted p-value<0.05. (D) Venn diagram showing enriched motifs in ATAC-seq peaks at gene's promoters. (E-G) SOX17 ChIP-seq fold enrichment over IgG control (DOX+: dark red) and ATAC-seq signals (DOX+: dark magenta; DOX−: light magenta) around NOTCH4, CDX2 and HEY1 genes. ATAC-seq signals are from one of the two replicates. (H) Quantitative ChIP analysis of SOX17 and H3K27ac at the CDX2 promoter (mean±SD, n=3 experiments). ****p<0.0001, two-way ANOVA, Tukey's multiple comparisons test. See also FIG. 11 and FIG. 12.

FIG. 5 demonstrates that SOX17 induces expression of arterial and HOXA genes in day 5 HE. (A) Schematic diagram of experiments. (B and C) qPCR analysis of arterial markers (EFNB2, DLL4, Notch4, HEY1, CXCR4, SOX17), venous (NR2F2), RUNX1, and HOX (HOXA and CDX2) genes in day 5 HE subpopulations. Results are mean±SD for three independent experiments; *p<0.05, **p<0.01, ***p<0.001 and ****p<0.0001, two-way ANOVA, Sidak's multiple comparisons test.

FIG. 6 demonstrates that SOX17 regulates AHE formation through NOTCH signaling activation and HOXA genes expression through CDX2. (A) Schematic diagram of experiments. SOX17 cells were differentiated with DOX, and treated with DAPT or transfected with siCDX2. After 2 days (day 5 of differentiation), two major HE subsets were assessed by flow cytometry and isolated for RT-PCR analysis. (B) and (C) Flow cytometric analysis of AHE formation in DOX or No DOX cultures with or without DAPT. Plots in (C) show the percentages and total number of cells generated from 10⁴ hESCs. (mean±SD and triplicated independent experiments). *p<0.05, ***p<0.001, and ****p<0.0001, two-way ANOVA, Sidak's and Tukey's multiple comparisons test. qPCR analysis of HOXA genes (D) and arterial markers (EFNB2, NOTCH1, NOTCH4, HEY1, SOX17), and CDX2 (C) in day 5 HE subpopulations treated with scramble siRNA (SC) or siCDX2. The silencing efficiency of CDX2 with siRNA is more than 75% as shown in (E), right graph. Results are mean±SD for three independent experiments; *p<0.05, **p<0.01, ***p<0.001 and ****p<0.0001, two-way ANOVA, Sidak's multiple comparisons test and t-test.

FIG. 7 shows a model of SOX17 action in HE. SOX17 upregulates expression of genes associated with NOTCH signaling and binds directly to the CDX2 promoter, upregulating CDX2 expression. These molecular events lead to the upregulation of HOXA gene expression and establishment of AHE with robust lympho-myeloid potential and DLL4⁺CXCR4⁺SOX17^{hi} phenotype resembling AHE at AGM region.

FIG. 8 depicts the generation and characterization of SOX17 knockout H9 ESCs by CRISPR (related to FIG. 1). (A) Schematic diagram of SOX17 knockout strategy. P1 and P2 are screening primers. (B) SOX17 deletion was confirmed by genomic PCR using P1/P2 primers, WT (wild type; 1,233 bp) and HO (Homozygote; 403 bp). (C) SOX17 knockout in day 5 differentiated cells confirmed by western blot. (D and E) Flow cytometric analysis of CD43⁺ HPs generated on day 9 of differentiation. Graphs show the percentages and total number of cells generated from 10⁴ hESCs. (mean±SD and triplicated independent experiments) **p<0.01 and ****p<0.0001, t-test. (F) Pie charts displaying composition of CD43⁺ subsets on day 9 of differentiation. Results are mean±SD and triplicated independent experiments. *p<0.05, **p<0.01, and ***p<0.001, t-test. (G) CFC potential on day 9 of differentiation. (H) T cell output from 10⁴ CD43⁺ cells collected on day 9 of differentiation (mean±SD, n=3 experiments). ***p<0.001 and ****p<0.0001, t-test. (I) and (J) Two different SOX17 knockout H9 hESC clones confirms reduced arterial specification and T cell potential in setting of SOX17 loss.

FIG. 9 depicts the generation of DOX-inducible SOX17 H9 hESC line (related to FIG. 2). (A) Schematic diagram of PiggyBac system used to generate iSOX17 H9 cells. (B) Expression of Venus reporter in undifferentiated iSOX17 cells with or without DOX. Scale bars are 200 μm. (C) Western blot confirms upregulation of SOX17 expression in undifferentiated cell with or without dox.

FIG. 10 demonstrates the effects of SOX17 on proliferation and apoptosis of AHE at day 5 of differentiation (related to FIG. 3). (A) and (B) Flow cytometric analysis of cell cycle in the AHE in DOX, No Dox, and SOX17 knockout cells. Representative dot plots (A) and mean±SD (B) of triplicated independent experiments are shown. *p<0.05, **p<0.01, ***p<0.001, and ****p<0.0001, one-way ANOVA Dunnett's multiple comparisons test. (C) and (D) flow cytometric analysis of cell apoptosis using annexin V staining in the AHE in DOX, No Dox, and SOX17 knockout cells. Representative dot plots (C) and mean±SD (D) of triplicated independent experiments are shown. **p<0.01 and ***p<0.001, one-way ANOVA Dunnett's multiple comparisons test.

FIG. 11 shows a qPCR analysis that confirms the upregulation of HOXA, arterial and retinoic acid signaling associated genes in day 4 HE following SOX17 overexpression (related to FIG. 4). (A) Schematic diagram of experiments. (B) qRT-PCR analysis. Results are mean±SD and triplicated independent experiments. ****p<0.0001, t-test.

FIG. 12 demonstrates that SOX17 knockout suppresses the expression of HOXA genes and arterial markers in day 5 HE subsets (related to FIG. 4). (A) Schematic diagram of experiments. (B and C) qRT-PCR analysis of arterial (EFNB2, DLL4, NOTCH4, CXCR4) (B) and HOXA genes (HOXA5, HOXA6, HOXA7, HOXA9, HOXA10) and CDX2 (C) in D5 HE subpopulations. Results are mean±SD for three independent experiments; *p<0.05, ***p<0.001, and ****p<0.0001, 2-way ANOVA, Sidak's multiple comparisons test.

FIG. 13 shows the FMO controls for flow cytometric analysis of AHE at Day 5 of differentiation. Representative flow cytometry contour plots of AHE specification on day 5 of differentiation. FMO control for VEC, CD73, CD43, DLL4 and CXCR4 were used to determine the background.

FIG. 14 demonstrates that overexpression of SOX17 in hematopoietic progenitors generated from day 4 hemogenic endothelium enhances their myeloid and T lymphoid potentials. (A) Schematic diagram of experiments. (B) CFC potential of HP collected following culture of day 4+5+5. (C) Graphs show the total number of T cells produced from 10⁴ hematopoietic progenitors (HPs) collected following culture of day 4+5+5. **p<0.01, ***p<0.001, and ****p<0.0001

### DETAILED DESCRIPTION

The generation of functional hematopoietic stem cell (HSC)-like cells from pluripotent stem cells (PSCs) has been a long-sought goal in hematology research. Previous efforts to generate cells with myeloid and T cell hematopoietic potential from human pluripotent stem cells (hPSCs) have produced few, if any, cells capable of engrafting in irradiated mice.

Recent advances in understanding the major bottlenecks in the derivation of engraftable hematopoietic cells and definitive lympho-myeloid progenitors from PSCs have identified deficiencies in NOTCH and HOXA signaling as major contributing factors to the observed functional deficiencies. However, the hierarchy of molecular events that are critical to establishing these programs is still poorly understood. In the present application, the inventors identified the transcription factor SOX17 as a critical upstream activator of these pathways. As is described in the Examples, the inventors generated SOX17-knockout and SOX17-inducible human hPSCs, and using cell biology and molecular profiling, they show that SOX17 activates HOXA and arterial programs in hemogenic endothelium (HE) and establishes definitive lympho-myeloid hematopoiesis. The inventors further show that SOX17 produces these effects through activation of NOTCH and CDX2 signaling.

Based on these findings, the inventors have devised a novel strategy for producing superior HE that expresses HOXA cluster genes and has enhanced T lymphoid potential. Importantly, these methods enable enhanced T cell production, which could facilitate the development of off-the-shelf immunotherapies derived from hPSCs.

### Methods:

The present invention provides methods of enhancing arterial specification of hemogenic endothelium in differentiating human pluripotent stem cells (hPSCs). The methods involve (a) introducing an inducible SOX17 transgene into a population of hPSCs; (b) culturing the hPSCs for at least two days under conditions to differentiate the hPSC into mesoderm cells; and (c) inducing expression of the SOX17 transgene in the mesoderm cells on at least about day two of differentiation, such that DLL4⁺CXCR4⁺ arterial hemogenic endothelium (AHE) cells are obtained.

As is described in the Examples, the inventors discovered that they could promote the arterial program and HOXA gene expression in hemogenic endothelium derived from hPSCs in vitro by overexpressing SOX17 at a specific time (i.e., starting at day 2) and for a defined period (i.e., 2-4 days) during differentiation. SOX17 is a member of the Sry-related high mobility group domain (SOX) family of transcription factors, and is key developmental regulator of endothelial and hematopoietic lineages. In the methods of the present invention, differentiating hPSCs are forced to overexpress SOX17 during the mesoderm differentiation by introducing an inducible SOX17 transgene into the population of mesodermal cells to facilitate formation of AHE with robust lymphomyeloid potential. In addition, inventors discovered that an SOX17 expands in suspension cultures lymphomyeloid progenitors generated from hemogenic endothelium. This method provided increased amounts of progenitors which could differentiate into lymphoid cells.

The SOX17 transgene used with the present invention may comprise any nucleic acid sequence encoding the SOX17 protein. For Example, the SOX17 transgene may be obtained by amplifying the SOX17 gene sequence from the genomic locus in human cells or by amplifying SOX17 mRNA from hPSCs differentiated into endothelial and blood cells and converting it into cDNA. Alternatively, genomic DNA or cDNA clones can be obtained commercially (e.g., from Sino Biological, Origene, IDT, etc.). In some embodiments, the transgene comprises SEQ ID NO:58, a cDNA sequence encoding the human SOX17 protein.

In some embodiments, the SOX17 transgene further comprises a vector sequence that can be used to drive the expression of the SOX17 transgene within the cells. In these embodiments, the transgene is introduced by into the population of hPSCs by transducing the cells with said vector. As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”. Vectors suitable for use with the present invention comprise a nucleotide sequence encoding a SOX17 transgene and a heterogeneous sequence necessary for proper propagation of the vector and expression of the encoded polypeptide. The heterogeneous sequence (i.e., sequence from a different species than the transgene) can comprise a heterologous promoter or heterologous transcriptional regulatory region that allows for expression of the polypeptide. Suitable vectors for the expression of the SOX17 transgene include plasmids and viral vectors. In a preferred embodiment, the vector comprises heterologous sequence that allows the transient and/or inducible expression of the encoded SOX17 protein.

In some embodiments, the vector includes a transposase system, such as the PiggyBac transposon system (see Examples). The PiggyBac transposon is a TTAA-specific mobile genetic element that efficiently transposes between vectors and chromosomes via a “cut and paste” mechanism. PiggyBac transposase recognizes transposon-specific inverted terminal repeat sequences (ITRs) and moves the intervening contents to a TTAA insertion site in a chromosome or another vector. Thus, inserting a gene of interest between two ITRs in a transposon vector allows one to efficiently insert the gene into a target genome. Other suitable transposase systems for use with the present invention include, for example, Sleeping Beauty.

In other embodiments, the vector is a plasmid, a viral vector, a cosmids, or an artificial chromosome. Suitable plasmids include, for example, E. coli cloning vectors. Many suitable viral vectors are known in the art and include, but are not limited to, an adenovirus vector; an adeno-associated virus vector; a pox virus vector, such as a fowlpox virus vector; an alpha virus vector; a baculoviral vector; a herpes virus vector; a retrovirus vector, such as a lentivirus vector; a Modified Vaccinia virus Ankara vector; a Ross River virus vector; a Sindbis virus vector; a Semliki Forest virus vector; and a Venezuelan Equine Encephalitis virus vector. In one particular embodiment, the vector comprises SEQ ID NO:1.

In some embodiments, the vector is an expression vector that comprises a promoter that drives the expression of the SOX17 transgene, preferably transient or inducible expression of the SOX17 transgene. As used herein, the term “promoter” refers to a DNA regulatory region capable of binding RNA polymerase in a cell and initiating transcription of a coding sequence. Although promoters are most commonly found immediately upstream of a coding sequence, they may also be found downstream of or within the coding sequence. Promoters may be derived in their entirety from a native gene or may be composed of multiple elements, including elements derived from promoters found in nature or elements comprising synthetic DNA sequences. It is understood by those skilled in the art that different promoters may direct the expression of a gene in different tissues or cell types, at different stages of development, or in response to different environmental conditions. Preferably, the promoters used with the present invention are inducible promoters. An “inducible promoter” is a promoter that is activated (i.e., initiates transcription) only in the presence of a particular molecule. Inducible promoters allow tight control the expression of a transgene within cells. Many suitable inducible expression systems are known in the art and include, for example, Tet-On gene expression systems that allow one to induce the expression of a gene by administering tetracycline (Tc) or tetracycline-derivatives like doxycycline (DOX). Suitable Tet-On systems for use with the present invention include, without limitation, Tet-On Advanced and Tet-On 3G. Tet-On systems utilize several promoters, including both minimal promoters (e.g., CMV) flanked by a tetracycline response element (TRE) and engineered Tet-inducible promoters (e.g., TRE2 and TREtight). For instance, in the Examples, the SOX17 transgene is inserted (i.e., via conventional cloning methods) downstream of the doxycycline-inducible TREtight promoter within a vector. This vector was introduced into the hPSCs, allowing the inventors to induce expression of the SOX17 transgene at the desired stage of differentiation by adding doxycycline to the cell culture to activate expression from the TREtight promoter. Those of skill in the art are aware of many additional inducible gene expression systems, including both chemical-inducible and temperature-inducible systems. Other suitable inducible gene expression systems for use with the present invention include, without limitation, the glucocorticoid-responsive mouse mammary tumor virus promoter (MMTVprom), the tamoxifen-responsive hormone-binding domain of the estrogen receptor (ER^{TAM}), the ecdysone-inducible promoter (EcP), heat shock inducible promoters (e.g., Hsp70 or Hsp90-derived promoters), and the T7 promoter/T7 RNA polymerase system (T7P). The SOX17 transgene may be introduced into the hPSCs using any suitable method, for example by transfection or transduction. In one embodiment, the transgene is introduced by transducing the hPSCs with a vector comprising the SOX17 transgene. In another embodiment, the hPSCs are transduced with an exogenous SOX17 mRNA. In yet another embodiment, the hPSCs are transduced with the SOX17 protein.

In the present methods, the hPSCs are cultured under conditions for at least two days to differentiate these cells into mesoderm cells. Notably, mesoderm cells can be identified by their KDR⁺ phenotype. Methods of differentiating hPSCs into progenitor mesoderm cells are known in the art. In one embodiment, mesoderm cells are obtained by culturing hPSCs in a chemically defined culture medium for about 2 days to about 4 days, whereby a cell population comprising mesoderm cells is obtained. For example, the hPSCs may be cultured in xenogen-free, serum-albumin free chemically defined medium comprising BMP4, activin A, LiCl, and FGF2, as described in Uenishi et al. (Stem cell reports (2014): 1073-1084) and U.S. Pat. No. 9,938,499, which is incorporated by reference in its entirety. For example, the chemically defined medium comprises about 10 ng/ml to about 50 ng/ml FGF2, about 50 ng/ml to about 250 mg/ml of BMP4 (e.g., 50 ng/ml to about 500 ng/ml BMP4), about 10 ng/ml to about 15 ng/ml Activin A, and about 1 to 2 mM LiCl under hypoxic conditions. In other embodiments, the cells are attached to a culture plate via extracellular matrix proteins. For example, in one embodiment, the cells are attached via collagena, fibronectin, matrigel™ or Tenascin C (TenC). In a preferred embodiment, the cells are cultured on plates coated with Collagen IV, as described in Uenishi et al. and U.S. Pat. No. 9,938.499.

The term “defined culture medium” is used herein to indicate that the identity and quantity of each medium ingredient is known. As used herein, the terms “chemically-defined culture conditions,” “fully defined, growth factor free culture conditions,” and “fully-defined conditions” indicate that the identity and quantity of each medium ingredient is known and the identity and quantity of supportive surface is known. As used herein the term “xenogen-free” refers to medium that does not contain any products obtained from a non-human animal source. As used herein, the term “serum albumin-free” indicates that the culture medium used contains no added serum albumin in any form, including without limitation bovine serum albumin (BSA) or any form of recombinant albumin. Standardizing culture conditions by using a chemically defined culture medium minimizes the potential for lot-to-lot or batch-to-batch variations in materials to which the cells are exposed during cell culture. Accordingly, the effects of various differentiation factors are more predictable when added to cells and tissues cultured under chemically defined conditions. As used herein, the term “serum-free” refers to cell culture materials that do not contain serum or serum replacement, or that contain essentially no serum or serum replacement. For example, an essentially serum-free medium can contain less than about 1%, 0.9%, 0.8%, 0.7%, 0.6%, 0.5%, 0.4%, 0.3%, 0.2%, or 0.1% serum. “Serum free” also refers to culture components free of serum obtained from animal blood and of animal-derived materials, which reduces or eliminates the potential for cross-species viral or prion transmission. Further, serum-containing medium is not chemically defined, producing a degree of variability in the culture conditions. Suitable defined media include, but are not limited to, E8 medium.

Expression of the SOX17 transgene may be induced using any suitable methods known in the art. For example, in embodiments in which the transgene is introduced as a vector comprising an inducible promoter operably linked to the SOX17 transgene, transgene expression can be induced by contacting the cells with the signaling molecule required for activation of the chosen promoter. In embodiments in which the transgene is introduced as SOX17 mRNA or protein, induction is accomplished by transducing with cells with the mRNA or protein at the appropriate stage of hPSC differentiation.

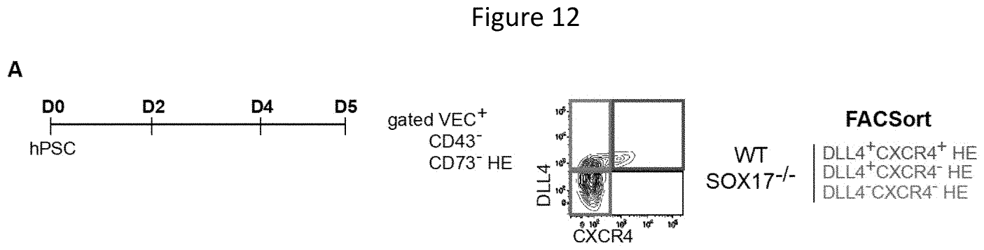

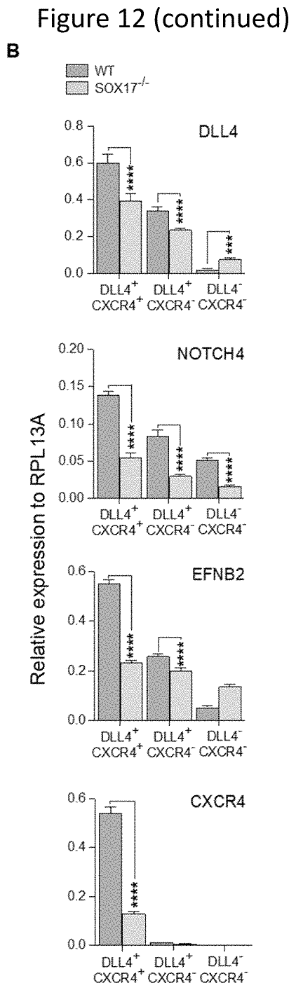

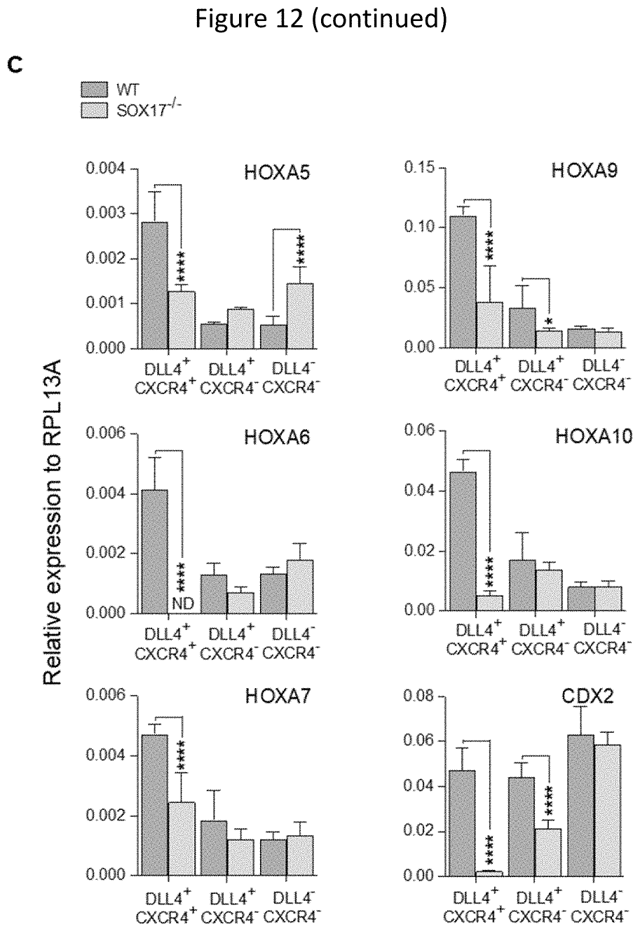

In a preferred embodiment, expression of the SOX17 transgene is induced at about day 2 of hPSC differentiation, i.e., in the mesoderm stage of development. In embodiments in which the transgene is provided as a vector, induction may involve treating the cells with the signaling molecule required to activate an inducible promoter (e.g., DOX). Alternatively, one can induce SOX17 expression by transfecting the hPSCs with mRNA encoding SOX17 or with the SOX17 protein at about day 2 of differentiation. The inventors discovered that inducing SOX17 expression for two to four days (i.e., from day 2 to day 4-6 of differentiation) generated the greatest number of CD43⁺/CD45⁺ cells (FIG. 12B-D), and produced cells with the greatest hematopoietic colony-forming potential. Thus, in preferred embodiments, the population of hPSCs expresses the SOX17 transgene for two to four days. In specific embodiments, the SOX17 transgene is expressed for at least two days, for at least three days, or for at least four days.

In another embodiment, SOX17 was upregulated in CD43⁺ hematopoietic progenitors generated from hemogenic endothelium on day 9 of hematopoietic differentiation to further expand lymphomyeloid progenitors. This additional SOX17 upregulation resulted in an greater expansion of lymphomyeloid progenitors when compared to the method without such upregulation.

The human pluripotent stem cells (hPSCs) used with the present methods may be embryonic stem cells (ESCs) or induced pluripotent stem cells (iPSCs). Many stem cells lines are commercially available. For example, the inventors utilized a H9 hESC (WA09) line from WiCell.

The goal of the present invention is to produce a population of arterial type hemogenic endothelial cells (AHEs) with broad lymphoid potential that are able to give rise to engraftable hematopoietic cells. These superior AHE cells can be identified as having the DLL4⁺CXCR4⁺ phenotype. CXCR4 is a HSC homing receptor that is not present in hematopoietic progenitors produced by traditional hESC differentiation methods, and DLL4 (Delta Like Canonical Notch Ligand 4) is a NOTCH-signaling ligand expressed by HE in vivo. In the Examples, the inventors show that by inducing SOX17 expression in differentiating hPSCs they produce a greater number of AHE cells with these desirable characteristics. Specifically, they found that SOX17 expression increases the production of AHE cells by about 50%.

As is demonstrated in the Examples, inducing the expression of SOX17 causes upregulation of genes encoding HOXA family members and arterial markers, which facilitates arterial specification of HE from mesoderm cells. In the present methods, hPSCs are cultured under conditions to differentiate the hPSC into mesoderm cells for about 4 days, and 24 hours later (on day 5) the HE begin to specify into DLL4⁺CXCR4^{+/-} arterial HE and non-arterial DLL4⁻ CXCR4⁻ HE. Enhanced arterial specification may be detected at day 4 and 5 as (1) an increase in the percentage of VEC⁺CD73⁻CD43⁻ HE cells generated, (2) an increase in the percentage of cells expressing DLL4 and CXCR4, which express increased levels of arterial marker genes (EFNB2, NOTCH4, HEY1, CXCR4, DLL4) and produce higher numbers of blood cells with colony forming cell (CFC) potential and/or T cell potential, as compared to the cells generated when SOX17 expression is not induced.

The inventors discovered that SOX17 expression enhances arterial specification via activation of several signaling pathways, including the Notch signaling pathways. Additionally, the inventors demonstrated that SOX17 is essential for the expression of HOXA genes in AHE. Thus, in some embodiments, the AHE cells produced by the methods disclosed herein express one or more HOXA genes selected from the group consisting of HOXA5, HOXA7, HOXA9, HOXA10, and HOX11. HOX genes have been implicated in hematopoiesis across species. For example, HOXA9 is the key homeotic gene that defines HSC identity, supporting HSC renewal during embryogenesis and stress hematopoiesis. HOXA5, HOXA9, and HOXA10 are transcriptional targets of Notch signaling in T cell progenitors, suggesting a role for these protein in T-lymphopoiesis. While SOX17 expression was previously known to be correlated with HOXA expression, it was not known to be causative. Thus, the inventors' discovery that SOX17 activates HOXA expression represents a significant advance in understanding the mechanisms of hematopoietic specification.

Further, the inventors have determined that SOX17 activates HOXA expression by binding to the promoter of CDX2 (a master regulator of HOX gene expression) and upregulating its expression. Thus, in some embodiments, the AHE cells produced by the methods disclosed herein express CDX2.

The AHE cells produced by the methods of the present invention have definitive lympho-myeloid potential and are, thus, useful for the production of myeloid and lymphoid progenitor cells. Those of skill in the art may consult a standard cell differentiation protocol (e.g., Stem cell reports (2014): 1073-1084; Nature protocols (2011): 296-313; Stem cells and development (2011): 1639-1647; and Blood (2005): 617-626) to obtain cell populations of the desired hematopoietic cell type. For example, the AHE of the present invention may be further differentiated into T cells, beta-hemoglobin-producing red blood cells, megakaryocytic cells, and multipotential myeloid progenitors, including granulocyte, erythrocyte, megakaryocyte, macrophage (GEMM) and granulocyte-macrophage (GM) colony forming cells (CFCs), and mature myelomonocytic cells. In some embodiments of the present methods, the cells are further differentiated into lymphoid cell lines.

In the Examples, the inventors demonstrate that DLL4⁺CXCR4⁺ AHE cells produced by the present methods have increased T cell potential, i.e., they give rise to an increased number of T cells. Specifically, in DOX-treated conditions, DLL4⁺CXCR4⁺ AHE cells generate 54-fold more CD4⁺CD8⁺ T cells as compared to DLL4⁻CXCR4⁻ HE cells. Scalable T cell production is essential to advance iPSC-based immunotherapies into the clinic. Thus, in preferred embodiments, the cells are further differentiated into T cells. T cells may be differentiated using known methods, including the method disclosed in the Examples (see the Materials and Methods section titled “T cell differentiation”). The T cells can be identified as CD4+CD8+. In some embodiments, the T cells are identified as CD7+CD5+, CD8+CD4+, or a combination thereof (CD7+CD5+ and CD8+/CD4+).

Suitably, in one embodiment, the day 4 HE or day 5 AHE cells were cultured on OP9-DLL4 in presence of TPO (about 20-70 ng/ml, e.g., 50 ng/ml), SCF (about 20-70 ng/ml, e.g., 50 ng/ml), IL-6 (about 10-40 ng/ml, e.g., 20 ng/ml), IL-3 (about 5-30 ng/ml, e.g. 10 ng/ml) and FLT3L (3-20 ng/ml, e.g., 10 ng/ml) for about 4-5 days to produce hematopoietic progenitors enriched in T cell potential which were differentiated into T cells by culturing in the presence of delta-like ligand 4, e.g., cells overexpressing DLL4 (DLL4-expressing cells, e.g., OP9-DLL4 cells) for a sufficient time to produce T cells (e.g., at least 2 weeks, preferably at least three weeks or more). The media further comprise about 5-20 ng/ml SCF, about 3-20 ng/ml FLT3L and about 3-20 ng/ml IL-7 to augment T cell production. For example, in one embodiment, the AHE cells are passaged weekly onto fresh OP9-DLL4 cells in media comprising about 10 ng/ml SCF, about 5 ng/ml FLT3L and about 5 ng/ml IL-7 for about 3 weeks. Cells can be analyzed by flow cytometry for T cell surface markers.

In another embodiment, the hematopoietic progenitors obtained from AHE cells are cultured in media comprising 5-50 ng/ml SCF, about 3-20 ng/ml FLT3L and about 3-20 ng/ml IL7 in the presence of DLL4-Fc (commercially available), DLL4-Fc ligands or on DLL4-OP9 cells for a sufficient time to differentiate into T cells.

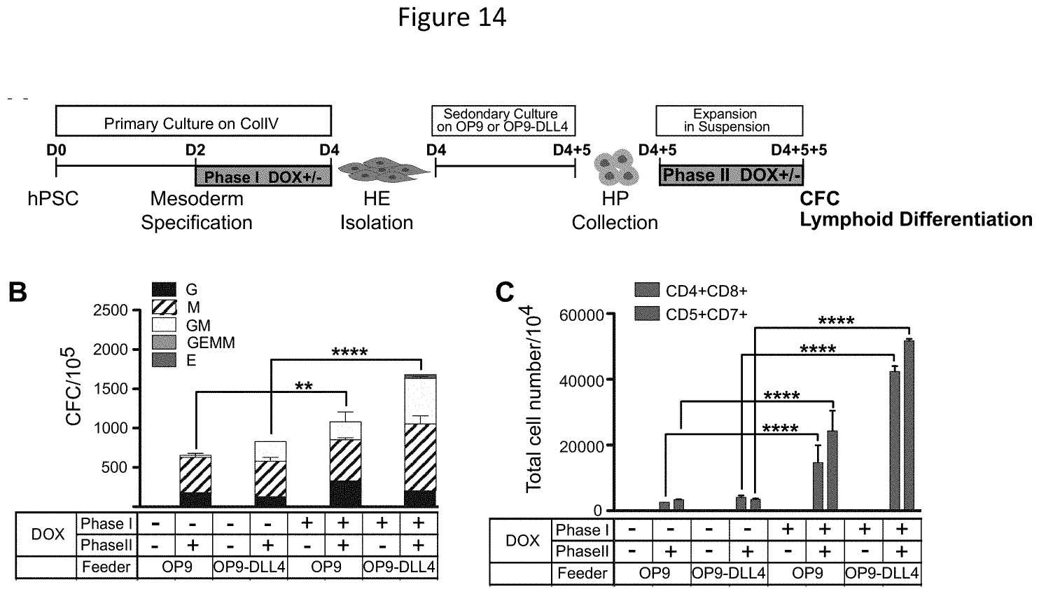

It was also surprisingly found that expansion of hemogenic progenitors (HP) which are subject to a second increased expression of SOX17 at day 4+5 through day 4+5+5 led to an increased expansion of myeloid and T lymphoid progenitors, as demonstrated in FIG. 14, which allows for the differentiation of a significant increase in the production of CD4+CD8+ T cells.

Thus, in another embodiment, CD43+ hematopoietic progenitors generated from day 4 hemogenic endothelium in coculture with OP9 or OP9-DLL4 in medium comprising 20-70 ug/ml TPO and SCF (e.g., 50 ug/ml), 10-30 ng/ml IL6 (e.g., 20 ng/ml), and 5-25 ng/ml IL3 and FLT3L (e.g., 10 ng/ml) for 5 days, were collected and cultured in low attachment plate with IF9S media with 50-150 ng/ml FLT3L (e.g. 100 ng/ml) FLT3L, TPO and SCF, and about 10-40 ng/ml (e.g., 20 ng/ml) IL6, 3-20 ng/ml, e.g. 10 ng/ml IL3, with 2 μM Doxycyclin to induce expression of SOX17. As demonstrated in FIG. 14, after 5 days, the cells were collected and demonstrated an enhanced myeloid CFC and T cell potentials. As demonstrated in Example 2, a second SOX17 overexpression in day 4+5 hematopoietic progenitors expanded the myeloid and T lymphoid progenitors and this expansion was more profound when day4 hemogenic endothelial cells were generated in presence of doxycycline (day 2-4 doxycycline treatment) and cultured on OP9-DLL4 (FIG. 14).

T cells produced by the methods disclosed herein may be used for the production of various therapeutics, including chimeric antigen receptors (CAR) T cells. CAR T cells are T cells that have been genetically engineered to produce an artificial T cell receptor (i.e., a CAR) that allows them to target a specific protein of choice. CARs comprise an antigen-specific recognition domain that binds to specific target antigen or cell and a transmembrane domain linking the extracellular domain to an intracellular signaling domain. CAR T cells are commonly designed to recognize cancer cells (e.g., via recognition of an antigen that is present on the tumor surface) for use in cancer immunotherapies. Thus, in one embodiment, the methods of the present invention further comprise using the AHE to generate CAR expressing T cells that can be used to kill tumor cells. Methods of designing and producing CART cells are known in the art.

### Compositions:

The present invention also encompasses cell populations produced by the methods disclosed herein. In some embodiments, the AHE cells are sorted from the cell culture, e.g., based on expression of DLL4 and CXCR4. The resulting hemogenic cell population will contain a SOX17 transgene, and may be at least 90%, 95% or 99% pure.

In another aspect, the present invention provides hPSC populations that comprise a SOX17 transgene and are capable of differentiating into DLL4⁺CXCR4⁺ arterial hemogenic endothelium (AHE) cells. In some embodiments, the hPSC cells comprise a vector comprising the SOX17 transgene. In some embodiments, said vector comprises an inducible promoter operably linked to the SOX17 transgene (e.g., the vector of SEQ ID NO:1 or a vector comprising SEQ ID NO:58), allowing for induction of SOX17 expression within the cells. Suitable vectors are discussed in the previous section.

Additionally, the present invention provides isolated in vitro populations of DLL4⁺CXCR4⁺ arterial hemogenic endothelium (AHE) cells differentiated from an hPSC population comprising a SOX17 transgene. As used herein, the phrase “isolated in vitro population” refers to a population of cells that is grown outside of a living organism (e.g., in a test tube, flask, or culture dish) under defined conditions.

These DLL4⁺CXCR4⁺ AHE cells may be further differentiated into T cells, forming an isolated in vitro T cell population. T cell differentiation may be accomplished using known methods, as discussed above. In some embodiments, the T cell population comprises more than 90% CD4⁺CD8⁺ T cells, alternatively at least 95% CD4⁺CD8⁺ T cells.

In some embodiments, the AHE derived herein can produce hematopoietic progenitors (HP) with a high T cells potential. As used herein, the phrase “hematopoietic progenitors with high T cell potential” refers to a population of HE cells that is able to differentiate into T cells at least 10 fold more efficiently.

Further, in some embodiments, the present invention provides hematopoietic progenitors (HP) derived from the AHE that have high myeloid and T cell potential, as demonstrated in Example 2. These cells are differentiated by a method comprising at least two induction of SOX17 expression, leading to a more robust production of cells with myeloid and T cell potential as compared to without SOX17 expression.

As is discussed in the previous section, T cells are useful for the production of therapeutics, including, for example, chimeric antigen receptors (CAR) T cells. Thus, in some embodiments, the T cells are engineered to express an exogenous chimeric antigen receptor (CAR).

The following non-limiting examples are included for purposes of illustration only, and are not intended to limit the scope of the range of techniques and protocols in which the compositions and methods of the present invention may find utility, as will be appreciated by one of skill in the art and can be readily implemented. The present invention has been described in terms of one or more preferred embodiments, and it should be appreciated that many equivalents, alternatives, variations, and modifications, aside from those expressly stated, are possible and within the scope of the invention.

### EXAMPLES

### Example 1

SOX17 has been implicated in arterial specification and the maintenance of hematopoietic stem cells (HSCs) in the murine embryo. However, knowledge about molecular pathways and stage-specific effects of SOX17 in humans remains limited. Here, using SOX17-knockout and SOX17-inducible human pluripotent stem cells (hPSCs), paired with molecular profiling studies, we reveal that SOX17 is a master regulator of HOXA and arterial programs in hemogenic endothelium (HE), and is required for the specification of HE with robust lympho-myeloid potential and DLL4⁺CXCR4⁺ phenotype resembling arterial HE at sites of HSC emergence. Along with activation of NOTCH signaling, SOX17 directly activates CDX2 expression leading to the upregulation of the HOXA cluster genes. Since deficiencies in NOTCH signaling and HOXA regulation were identified as major contributing factors to the impaired engraftment potential of hPSC-derived hematopoietic cells, identification of SOX17 as a key regulator linking arterial and HOXA programs in HE may help to program the HSC fate from hPSCs.

### Background:

Sox17 has been found to be expressed in the arterial vasculature (Liao et al., 2009) and the hemogenic endothelium (HE) in aorta-gonad-mesonephros (AGM) region (Clarke et al., 2013; Corada et al., 2013), in which it is required for arterial specification (Corada et al., 2013) and essential for establishing the definitive, but not primitive, hematopoietic program (Clarke et al., 2013) within the murine embryo. Although Sox17 actively prevents endothelial-to-hematopoietic transition (EHT) by repressing Runx1 (Lizama et al., 2015), Sox17 remains critical for maintaining intra-aortic hematopoietic clusters (IAHC) and fetal liver HSCs (Kim et al., 2007; Nobuhisa et al., 2014; Saito et al., 2018). Transduction of human embryonic stem cell (hESC)-derived CD34⁺ HE/OP9 cocultures with a tamoxifen-inducible murine Sox17 transgene revealed that tamoxifen treatment expands CD34⁺CD43⁺CD45^{-/low} cells co-expressing the endothelial marker VE-cadherin (VEC) (Nakajima-Takagi et al., 2013). Although these expanded cells possessed the capacity to form compact colonies in hematopoietic CFC medium with SCF, TPO and IL3, they were interpreted as HE cells. In mouse studies, the effects of Sox17 were attributed to the activation of the NOTCH signaling pathway by its direct binding to Dll4, Notch1 and Notch4 loci (Clarke et al., 2013; Corada et al., 2013). However, no activation of NOTCH pathway following Sox17 overexpression was observed during hESC differentiation (Nakajima-Takagi et al., 2013). While these studies established an important role of SOX17 in specification of definitive hematopoiesis and its diverse effects on EHT and HSCs, the molecular program induced by SOX17 at distinct stages of hematopoietic development, especially in humans, remains poorly understood.

To define the mechanisms of SOX17 action during specification and diversification of HE, we established SOX17-knockout and SOX17-inducible hESC lines and assessed their differentiation in a 2D chemically defined, feeder- and xeno-free human pluripotent stem cell (hPSC) differentiation system in which all stages of hematopoietic development are temporally, phenotypically, and functionally defined (Uenishi et al., 2014). In this study we specifically focused on the earliest stages of HE emergence and its arterial specification which have not been previously assessed. We reveal that SOX17 is required for the activation of HOXA expression and establishing arterial-type HE (AHE) with robust lympho-myeloid potential that can be identified by DLL4⁺CXCR4⁺ phenotype resembling AHE at sites of HSC emergence in vivo. Furthermore, the SOX17 effects are mediated by CDX2. These findings are important for understanding the molecular mechanisms controlling HE and definitive blood lineage development and designing strategies for specifying HSC fate from hPSCs.

### Materials and Methods:

### Generation Inducible SOX17 H9 ESC Line and SOX17 Knockout H9 ESC Line

We generated an inducible SOX17 cell line using the PiggyBac system (Park et al., 2018a). Human SOX17 CDS was cloned into PiggyBac transposon vector (Transposagen) downstream of TREtight promoter of pTRE-P2A-Venus-rpEF1a-Zeo plasmid, and cotransfected with pEF1α-M2rtTA-T2A-Puro and transposase plasmid into H9 hESCs using human stem cell nucleofector kit 2 (Lonza). Cells were selected in Zeocin (0.5 μg/ml, Thermofisher) and Puromycin (0.5 μg/ml, Sigma) for 10 days and resistant clones screened for Venus expression following DOX (Sigma) treatment. To generate SOX17^{-/-} knockout H9 ESC line, two single guide RNAs were designed in CRISPR design tool (Synthego). The two sgRNA sequences are provided as SEQ ID NO:2 and SEQ ID NO:3. H9 ESCs were electroporated with the two sgRNAs and Cas9 protein (PNA Bio), and then plated at a low density on 6 well plate. After 7 days, individual colonies were picked and further expanded. After expansion, individual clones were screened by genomic PCR for the acquisition of 830 bp deletion in wildtype SOX17 allele using primers P1 and P2.

hESC Lines Maintenance and Hematopoietic Differentiation

hPSCs (H9 hESC (WA09) line from WiCell), iSOX17 H9 line and knockout SOX17 H9 line were maintained and passaged on Matrigel in mTeSR1 media (WiCell). The cell lines were differentiated on collagen IV (ColIV)-coated plate (Uenishi et al., 2014). Cell lines were plated at a density of 5,000 cells/cm² onto 6 well plates with E8 media containing 10 μM Rock inhibitor (Y-27632, Cayman Chemicals). The following day, the media was changed to IF9S media with 50 ng/ml FGF2 (PeproTech), 50 ng/ml BMP4 (PeproTech), 15 ng/ml Activin A (PeproTech), and 2 mM LiCl (Sigma), and cultured in hypoxia (5% CO₂, 5% O₂). On day 2, the media was changed to IF9S media with 50 ng/ml FGF2, 50 ng/ml VEGF (PeproTech), and 2.5 μM TGF-β inhibitor (SB-431542, Cayman), and cultured in hypoxia (5% CO₂, 5% O₂). On days 4 and 6, the media was changed to IF9S media with 50 ng/ml FGF2, 50 ng/ml VEGF, 50 ng/ml TPO (PeproTech), 50 ng/ml IL-6 (PeproTech), 20 ng/ml SCF (PeproTech), and 10 ng/ml IL-3 (PeproTech), and cultured in normoxia (20% CO₂, 5% O₂). DOX (Sigma) was added to cultures on day 2 of differentiation at concentration of 2 μg/ml.

### Hemangioblast (HB)-CFC and Hematopoietic CFC Assay

HB-CFCs were detected using a semisolid colony-forming serum-free medium (CF-SFM) containing 40% ES-Cult M3120 methylcellulose (2.5% solution in IMDM, Stem Cell Technologies), 25% StemSpan serum-free expansion medium (SFEM, Stem Cell Technologies), 25% human endothelial serum-free medium (ESFM, ThermoFisher), 10% BIT 9500 supplement (Stem Cell Technologies), GlutaMAX (1/100 dilution, ThermoFisher), Ex-Cyte (1/1000 dilution, Millipore), 100 μM MTG, 50 μg/ml ascorbic acid and 20 ng/ml FGF (Peprotech) (Vodyanik et al., 2010). Hematopoietic CFCs were detected using serum containing H4435 MethoCult with FGF, SCF, IL-3, IL-6 and EPO (Stem Cell Technologies) following plating 1000 CD43⁺ cells/dish in duplicates. CFCs numbers recalculated per 10⁵ cells.

### Isolation and Culture of D4 HE

Immature/primordial HE cells were isolated from knockout SOX17 or DOX+ and DOX− iSOX17 differentiation cultures by CD31 MACS (Miltenyi Biotec) at D4. Isolated cells were plated on OP9 or OP9-DLL4 in α-MEM (Gibco) with 10% FBS (Hyclone) with TPO, SCF (50 ng/ml), IL-6 (20 ng/ml), IL-3 and FLT3L (10 ng/ml; all from Peprotech). The media was changed 24 hours later, and extra media was added another 2 days later. After 5 days in secondary culture, cells were collected and assessed for CFC and T cell potential.

### Isolation and Culture of D5 HE and Limiting Dilution Assay (LDA)

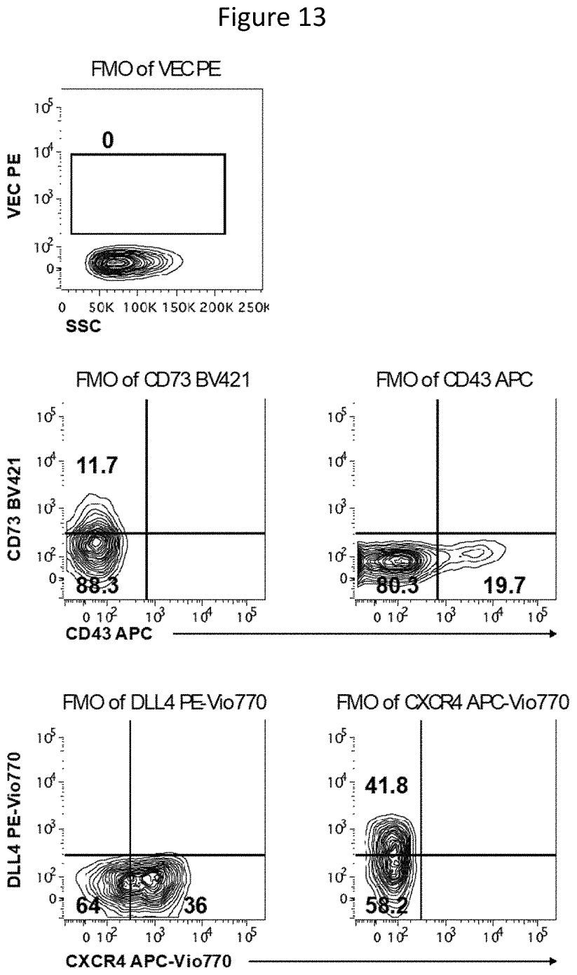

H9, iSOX17 and SOX17^{-/-} ESCs were collected on day 5 of differentiation, singularized by 1× TrypLE, and stained for VEC (CD144), CD73, CD43, DLL4, CXCR4 with dead cells excluded using Ghost Dye Violet 540 (Tonbo Biosciences). FMO controls for flow cytometric analysis are shown in FIG. 13. VEC⁺CD73⁻CD43⁻DLL4⁻CXCR4⁻, VEC⁺CD73⁻CD43⁻ DLL4⁺CXCR4⁻, VEC⁺CD73⁻CD43⁻DLL4⁺CXCR4⁺ and VEC⁺CD73⁻CD43⁻DLL4⁻CXCR4⁺ subsets were isolated using a FACSAria II cell sorter (BD Biosciences) and MA900 cell sorter (Sony Biotechnology) and were plated on OP9 or OP9-DLL4 at 20,000 cells/well of a 12-well plate in α-MEM media with 10% FBS (Hyclone) with TPO, SCF (50 ng/ml), IL-6 (20 ng/ml), IL-3 and FLT3L (10 ng/ml). On the next day, the media was changed and extra media was added 2 days later. The floating CD43⁺ cells were collected after 5 days of secondary culture and used for T cell and CFC assay. LDAs were conducted with sorted cells from day 5 differentiation cultures (no dox: DLL4⁺CXCR4⁻ HE and dox: DLL4⁺CXCR4⁻ HE or DLL4⁺CXCR4⁺ HE) following culture on DLL4-OP9 for 8 days. Row A of a 96-well plate received 3 cells/well, and each subsequent row afterward had twice the previous row (Row B contained 7, Row C contained 15 . . . Row H contained 500 cells). Eight days later, the cells were fixed and stained for immunofluorescent staining with anti-CD43 PE and DAPI in order to score the hematopoietic colonies using immunofluorescence microscopy. Extreme limiting dilution analysis was conducted using a previously established algorithm (Hu and Smyth, 2009).

Expansion of Hematopoietic Progenitors.

On day 4, HE cells were isolated from DOX+ or DOX− cultures by CD31 MACS. Isolated cells were plated on OP9 or OP9-DLL4 in α-MEM with 10% FBS with 50 ug/ml TPO and SCF, 20 ng/ml IL6, and 10 ng/ml IL3 and FLT3L. The media was changed 24 hours later, and extra media was added another 2 days later. After 5 days in in secondary culture, floating cell were collected and plated lin ow attachment 24 well plate. Cell were cultured with IF9S media with 100 ng/ml FLT3L, TPO and SCF, 20 ng/ml IL6, 10 ng/ml IL3, and with or without 2 μM Doxycyclin, and extra media was added another 2 days later. After 5 days, cells were collected and assessed for CFC and T cell potential.

### T Cell Differentiation

Floating hematopoietic cells were collected from day 9 differentiation cultures or day 5 secondary OP9 or OP9-DLL4 cocultures (D4 HE+5 or D5 HE +5), and were cultured on OP9-DLL4 in α-MEM with 20% FBS, 10 ng/ml SCF, 5 ng/ml FLT3L and IL-7 (PeproTech) on OP9-DLL4 for 3 weeks. Cells were passaged weekly onto fresh OP9-DLL4 cells. Cells were analyzed by flow cytometry for T cell surface markers after 21 days.

DAPT Treatment and CDX2 Knockdown in Differentiation Cultures Using siRNA

Notch signaling was blocked by DAPT ( [symbol] -secretase inhibitor, 10 μM, Cayman Chemical) added on day 3 of differentiation. For knockdown of CDX2 expression, DOX-treated iSOX17 cells were transfected with 100 nM CDX2 siRNA SMARTpool (Dharmacon) or Scramble negative control siRNA (Dharmacon) on day 3 of differentiation using Lipofectamine RNAiMAX (ThermoFisher). Next day, differentiation media was replaced with fresh media and cells were harvested at day 5 of differentiation.

### Apoptosis and Cell Cycle Analysis

Apoptosis was detected by flow cytometry using Annexin V (BD). For cell-cycle analysis, D5 cells were incubated in culture medium with BrdU (10 μM, BD Pharmingen) for 2 hours and stained with antibodies. For BrdU detection, the BrdU flow kit with 7 AAD was used and performed per the manufacturer's instructions. Fluorescent reagents used for analysis, cell viability, apoptosis, and proliferation are listed in Table 1.

TABLE 1
Key resource information

| | | |
|---|---|---|
| REAGENT or RESOURCE | SOURCE | IDENTIFIER |

Antibodies

| | | |
|---|---|---|
| Annexin V-APC | BD Biosciences | Cat# 550474; RRID: AB_2868885 |
| BrdU BV450 (clone; 3D4) | BD Biosciences | Cat# 560810; RRID: AB_2033930 |
| CD4 APC (clone: RPA-T4) | BD Biosciences | Cat# 555349; RRID: AB_398593 |
| CD5 APC (clone: UCHT2) | BD Biosciences | Cat# 555355; RRID: AB_398594 |
| CD5 APC-Vio770 (clone: REA782) | Miltenyi Biotec | Cat# 130-111-110; RRID: AB_2658602 |
| CD5 PE-Vio770 (clone: REA782) | Miltenyi Biotec | Cat# 130-111-109; RRID: AB_2658600 |
| CD7 FITC (clone: M-T701) | BD Biosceinces | Cat# 555360; RRID: AB_395763 |
| CD7 PE (clone: CD7-6B7) | Miltenyi Biotec | Cat# 130-123-247; RRID: AB_2802013 |
| CD7 PE-Vio770 (clone: CD7-6B7) | Miltenyi Biotec | Cat# 130-105-901; RRID: AB_2659106 |
| CD8 PE (clone: HIT8a) | BD Biosceinces | Cat# 555635; RRID: AB_395997 |
| CD31 MicroBeads | Miltenyi Biotec | Cat# 130-091-935 |
| CD41a APC (clone: HIP8) | BD Biosceinces | Cat# 559777; RRID: AB_398671 |
| CD43 BV510 (clone: 1G10) | BD Biosceinces | Cat# 563377; RRID: AB_2722767 |
| CD43 PE (clone: 1G10) | BD Biosceinces | Cat# 560199; RRID: AB_1645655 |
| CD43 PE-Vio770 (clone: DF-T1) | Miltenyi Biotec | Cat# 130-099-763; RRID: AB_2658133 |
| CD43 APC (clone: DF-T1) | Miltenyi Biotec | Cat# 130-097-367; RRID: AB_2658128 |
| CD43 APC-Vio770 (clone: DF-T1) | Miltenyi Biotec | Cat# 130-101-174; RRID: AB_2658135 |
| CD45 BV421 (clone: HI30) | BD Biosceinces | Cat# 563879; RRID: AB_2744402 |
| CD73 APC (clone: AD2) | BD Biosceinces | Cat# 560847; RRID: AB_10612019 |
| CD73 BV421 (clone: AD2) | BD Biosceinces | Cat# 562430, RRID: AB_11153119 |
| CD73 PE (clone: AD2) | BD Biosceinces | Cat# 550257; RRID: AB_393561 |
| CD73 PE-Vio770 (clone: AD2) | Miltenyi Biotec | Cat# 130-120-795; RRID: AB_2752200 |
| CD144 APC (clone: REA199) | Miltenyi Biotec | Cat# 130-100-708; RRID: AB_2655155 |
| CD144 BV421 (clone: 55-7H1) | BD Biosceinces | Cat# 565670; RRID: AB_2744284 |
| CD144 BV605 (clone: 55-7H1) | BD Biosceinces | Cat# 743705; RRID: AB_2741685 |
| CD144 FITC (clone: REA199) | Miltenyi Biotec | Cat# 130-100-742; RRID: AB_2655151 |
| CD144 PE (clone: REA199) | Miltenyi Biotec | Cat# 130-118-495; RRID: AB_2751528 |
| CD144 PE-Vio770 (clone: 12G5) | Miltenyi Biotec | Cat# 130-100-720; RRID: AB_2655158 |
| CD184 APC (clone: 12G5) | BD Biosceinces | Cat# 555976; RRID: AB_398616 |
| CD184 APC-Vio770 (clone: | Miltenyi Biotec | Cat# 130-116-521; RRID: AB_2727587 |
| REA649) | | |
| CD184 BV421 (clone: 12G5) | BD Biosceinces | Cat# 562448; RRID: AB_11153865 |
| CD235a APC (clone: GA-R2 | BD Biosceinces | Cat# 551336; RRID: AB_398499 |
| (HIR2)) | | |
| CD309 PE (clone: 89106) | BD Biosceinces | Cat# 560494; RRID: AB_1645503 |
| CD309 PE-Vio770 (clone: | Miltenyi Biotec | Cat# 130-117-986; RRID: AB_2733181 |
| REA1046) | | |
| DLL4 APC (clone: MHD4-46) | Miltenyi Biotec | Cat# 130-096-560; RRID: AB_10827749 |
| DLL4 PE (clone: MHD4-46) | Miltenyi Biotec | Cat# 130-096-567; RRID: AB_10831209 |
| DLL4 PE-Vio770 (clone: MHD4- | Miltenyi Biotec | Cat# 130-101-587; RRID: AB_2651569 |
| 46) | | |
| GAPDH | Santa Cruz | Cat# SC-25778; RRID: AB_10167668 |
| | Biotechnology | |
| Goat IgG | R&D | Cat# AB-108-C; RRID: AB_354267 |
| Goat IgG HRP | Santa Cruz | Cat# SC-2354; RRID: AB_628490 |
| | Biotechnology | |
| H3K27ac | Millipore Sigma | Cat# 07-360; RRID: AB_310550 |
| Mouse IgG HRP | Santa Cruz | Cat# SC-2005; RRID: AB_631736 |
| | Biotechnology | |
| SOX17 | R&D | Cat# AF1924; RRID: AB_355060 |
| 7AAD | BD PharMingen | Cat# 559925; RRID: AB_2869266 |

Chemicals, peptides, And recombinant proteins

| | | |
|---|---|---|
| Recombinant Human/Murine/Rat | PeproTech | Cat# 120-14E |
| Activin A | | |
| Recombinant Human BMP4 | PeproTech | Cat# 120-05ET |
| Recombinant Human FGF-basic | PeproTech | Cat# 100-18B |
| Recombinant Human VEGF165 | PeproTech | Cat# 100-20 |
| Recombinant Human IL-6 | PeproTech | Cat# 200-06 |
| Recombinant Human IL-3 | PeproTech | Cat# 200-03 |
| Recombinant Human SCF | PeproTech | Cat# 300-07 |
| Recombinant Human TPO | PeproTech | Cat# 300-18 |
| Recombinant Human Flt3-Ligand | PeproTech | Cat# 300-19 |
| Recombinant Human IL-7 | PeproTech | Cat# 200-07 |
| LiC1 | Sigma | Cat# L9659 |
| SB431542 (TGF-13 inhibitor) | Cayman Chemical | Cat# 13031 |
| Collagen IV | Sigma | Cat# C5533 |
| Y-27632 (Rock inhibitor) | Cayman Chemical | Cat# 10005583 |
| Doxycycline hyclate | Sigma | Cat# D9891 |
| DAPI | Sigma | Cat# D8417 |
| DAPT (γ-secretase inhibitor) | Cayman Chemical | Cat# 13197 |
| Cas9 protein | PNA Bio | Cat# CP01 |
| Lipofectamine RNAiMAX | ThermoFisher | Cat# 13778150 |

Critical commercial assays

| | | |
|---|---|---|
| BrdU KIT | BD PharMingen | Cat# 559619; RRID: AB_2617060 |
| Human stem cell nucleofector kit2 | Lonza | Cat# VPH-5022 |
| EZ-Magna ChIP AIG Chromatin | Sigma | Cat# 17-10086 |
| Immunoprecipitation kit | | |
| ATAC-SEQ | Active Motif | Cat# 25079 |

Deposited data

| | | |
|---|---|---|
| RNA-seq, ATAC-seq, ChIP-seq | This study | GEO: GSE140341 |

Experimental models: cc/Hines

| | | |
|---|---|---|
| WA09 (H9) human ES cell | WiCell | Cat# RB66492 |
| pTRE-SOX17-P2A-Venus-rpEF1a- | This paper | N/A |
| Zeo and pEF1α-M2rtTA-T2A-Puro | | |
| (PiggyBac) H9 hESC line | | |
| SOX17-/- H9 hESC line | This paper | N/A |

Oligonucleotides

| | | |
|---|---|---|
| Primers of RT-qPCR, gPCR, and | This paper | N/A |
| ChIP-PCR, See Table S1 | | |
| sgRNA targeting seq. for SOX17 #1 | Synthego | N/A |
| GTTCATCGGCCGCCGGATAC | | |
| (SEQ ID NO: 2) | | |
| sgRNA targeting seq. for SOX17 #2 | Synthego | N/A |
| TTCACCTGCTTGCGCCGCCG | | |
| (SEQ ID NO: 3) | | |
| ON-TARGETplus human CDX2 | Dharmacon | Cat# L-015636-00-0010 |
| siRNA SMARTpool | | |
| ON-TARGETplus Non-targeting | Dharmacon | Cat# D-001810-10-05 |
| Control pool | | |

Recombinant DNA

| | | |
|---|---|---|
| pTRE-SOX17-P2A-Venus-rpEF1a- | This paper | N/A |
| Zeo | | |
| pEF1α-M2rtTA-T2A-Puro | This paper | N/A |
| Super piggyBac transposase | Transposagen | Cat# SPB-DNA |
| expression vector | | |

Software and algorithms

| | | |
|---|---|---|
| Prism versions 8 | GraphPad Software | www.graphpad.com/scientific-software/prism/ |
| | Inc. | |
| FlowJo 8.8.6 | FlowJo | www.flowjo.com |
| STAR (version 2.5.2b) | PMID: 23104886 | github.com/alexdobin/STAR |
| | (Dobin et al., | |
| | 2013) | |
| RSEM (version 1.3.0) | PMID: 21816040 | deweylab.github.io/RSEM/ |
| | (Li and | |
| | Dewey, 2011) | |
| DESeq2 (version 1.22.2) | DESeq2 (version | bioconductor.org/packages/release/bioc/html/ |
| | 1.22.2) | DESeq2.html |
| MEME (version 5.0.4) | PMID: 22610855 | meme-suite.org |
| | (Bailey and | |
| | Machanick, 2012) | |
| BWA (version 0.7.15) | PMID: 19451168 | github.com/lh3/bwa |
| | (Li and | |
| | Durbin, 2009) | |
| MACS2 (version 2.1.0) | PMID: 18798982 | github.com/macs3-project/MACS |
| | (Zhang et al., | |
| | 2008) | |

### Western Blot Assay

Cell extracts were prepared by adding IP Lysis buffer (ThermoFisher) with protease inhibitor cocktail (Sigma). Cell lysates (10 μg) were separated by Mini-protean TGX gels (Bio-rad). The separated proteins were transferred to a PVDF membrane, and were stained with antibodies for SOX17 (R&D) and GAPDH (Santa Cruz). Immunoblots were visualized using the ECL PLUS detection kit (Amersham Pharmacia) and analyzed using ChemiDox XRS+ Image Lab Software Version 5.2.1 (Bio-Rad).

Real-Time qPCR

RNA was extracted using the RNeasy Plus Micro Kit (Qiagen). RNA was reverse-transcribed into cDNA using random hexamer primers (Qiagen) with SMART MMLV reverse transcriptase (TaKaRa). qPCR was conducted using TB Green Advantage qPCR Premix (TaKaRa). RPL13A was used as the reference gene to normalize the data. Primer sequences are listed in Table 2.

TABLE 2
Primers used for RT-qPCR.00

| Gene | Forward Sequence (5′-3′) | Reverse Sequence (5′-3′) | Purpose |
|---|---|---|---|
| ALDH1A2 | TCTGTCCCTCTCTGCTTTCT | CTGCCTGGCCTACATGTTATC | qPCR |
| | (SEQ ID NO: 4) | (SEQ ID NO: 5) | |
| CDX2 | CCGAACAGGGACTTGTTTAGAG | AGGTTGGCTCTGGCATTTATAG | qPCR |
| | (SEQ ID NO: 6) | (SEQ ID NO: 7) | |
| CXCR4 | TCAGTGGCTGACCTCCTCTT | CTTGGCCTTTGACTGTTGGT | qPCR |
| | (SEQ ID NO: 8) | (SEQ ID NO: 9) | |
| DLL4 | CAGTGGGCAGCGAAGCTACA | ACAGGCAGTGGTAGCCATCCTC | qPCR |
| | (SEQ ID NO: 10) | (SEQ ID NO: 11) | |
| EFNB2 | CTCCTCAACTGTGCCAAACCA | GGTTATCCAGGCCCTCCAAA | qPCR |
| | (SEQ ID NO: 12) | (SEQ ID NO: 13) | |
| HEY1 | GGACTATCGGAGTTTGGGATTT | TGGGAAGCGTAGTTGTTGAG | qPCR |
| | (SEQ ID NO: 14) | (SEQ ID NO: 15) | |
| HEY2 | TTCAAGGCAGCTCGGTAACTGAC | CATACTGATGCACTGCTGGATGG | qPCR |
| | (SEQ ID NO: 16) | (SEQ ID NO: 17) | |
| HOXA1 | CCAACAGAAACATGCCAGAAG | CTCGCCTTTCGCTATATCCTAC | qPCR |
| | (SEQ ID NO: 18) | (SEQ ID NO: 19) | |
| HOXA2 | GATGCAGTTTCACCCAGTTTG | GATTGTGGTGAGTGTGTCTGTA | qPCR |
| | (SEQ ID NO: 20) | (SEQ ID NO: 21) | |
| HOXA3 | GATGGAGCCATGGGAAGATTAC | CATAGGGAGGAGGCTGAGATATAG | qPCR |
| | (SEQ ID NO: 22) | (SEQ ID NO: 23) | |
| HOXA4 | GGAGAAGGAGTTCCACTTCAAT | GGTCTTTCTTCCACTTCATCCT | qPCR |
| | (SEQ ID NO: 24) | (SEQ ID NO: 25) | |
| HOXA5 | GTTCCTGTCTCAATAGCTCCAA | GTGTCTCATCAAGTCACCTCTAC | qPCR |
| | (SEQ ID NO: 26) | (SEQ ID NO: 27) | |
| HOXA6 | AAAGCACTCCATGACGAAGG | GTCTGGTAGCGCGTGTAGGT | qPCR |
| | (SEQ ID NO: 28) | (SEQ ID NO: 29) | |
| HOXA7 | AGGTCCAGGATCAGGGTATT | CCAGAGAAGGAGGGATTGATTC | qPCR |
| | (SEQ ID NO: 30) | (SEQ ID NO: 31) | |
| HOXA9 | GCGCCTTCTCTGAAAACAAT | CAGTTCCAGGGTCTGGTGTT | qPCR |
| | (SEQ ID NO: 32) | (SEQ ID NO: 33) | |
| HOXA10 | GCAAAGAGTGGTCGGAAGAA | CGCTCTCGAGTAAGGTACATATTG | qPCR |
| | (SEQ ID NO: 34) | (SEQ ID NO: 35) | |
| HOXA11 | TGGTCTGGGACTCTCTTGAT | GGTCCCAAACCTGTCATTCT | qPCR |
| | (SEQ ID NO: 36) | (SEQ ID NO: 37) | |
| HOXA13 | CTGGAACGGCCAAATGTACT | GCTTCTTTCTCCCCCTCCTA | qPCR |
| | (SEQ ID NO: 38) | (SEQ ID NO: 39) | |
| LFNG | GCAACGTGGTCATCACAAAC | CTCGATGAAGCGGTCATACTC | qPCR |
| | (SEQ ID NO: 40) | (SEQ ID NO: 41) | |
| NOTCH1 | CAATGTGGATGCCGCAGTTGTG | CAGCACCTTGGCGGTCTCGTA | qPCR |
| | (SEQ ID NO: 42) | (SEQ ID NO: 43) | |
| NOTCH4 | AGCTCTGGAAAGAGGGTTTAAG | CTCCTGTGGCCTGTCTTATTT | qPCR |
| | (SEQ ID NO: 44) | (SEQ ID NO: 45) | |
| NR2F2 | TGGTTCCAAACCAGTTTATTCTGT | AAGTGCGTTTCCATCATCTTTGAG | qPCR |
| | (SEQ ID NO: 46) | (SEQ ID NO: 47) | |
| RARγ | ATGACCGGAACAAGAAGAAGAA | TTGCTGACCTTGGTGATGAG | qPCR |
| | (SEQ ID NO: 48) | (SEQ ID NO: 49) | |
| RPL13A | CCTGGAGGAGAAGAGGAAAGAGA | TTGAGGACCTCTGTGTATTTGTCAA | qPCR |
| | (SEQ ID NO: 50) | (SEQ ID NO: 51) | |
| SOX17 | GCCAAGGGCGAGTCCCGTA | GCATCTTGCTCAACTCGGCGTTGTGCA | qPCR |
| | (SEQ ID NO: 52) | (SEQ ID NO: 53) | |
| P1/P2 | GGGTACGCTGTAGACCAGAC | TTCAGCCGCTTCACCTGCTT | Genomic |
| | (SEQ ID NO: 54) | (SEQ ID NO: 55) | PCR |
| CDX2 | ACTCCCAAAGCAGTTGGATG | GCTTTTTTATGGCCCAGGCTG | ChIP-PCR |
| promoter | (SEQ ID NO: 56) | (SEQ ID NO: 57) | |

### RNA-Seq

One hundred nanograms of total RNA was used to prepare sequencing libraries using the Ligation Mediated Sequencing (LM-Seq) protocol, according to the paper guidelines (Hou et al., 2015) and quantified with the Qubit fluorometer (ThermoFisher). Final cDNA libraries were quantitated with the Qubit Fluorometer (ThermoFisher), multiplexed, loaded at a final concentration of 2.5 nM, and sequenced as single reads on the Illumina HiSeq 3000 (Illumina).

### ChIP-seq

Chromatin immunoprecipitation (ChIP) analysis of day 4 HE was performed, as described in the protocol included in the EZ-Magna ChIP A/G Chromatin Immunoprecipitation Kit (Millipore Sigma). Five nanograms of IP or control DNA was used to prepare sequencing libraries using the TruSeq ChIP Sample Preparation Kit (Illumina) as per the manufacturer instructions and quantified with the Qubit fluorometer (Life Technologies). All six TruSeq ChIP indexed samples were pooled per lane, loaded at a final concentration of 2.5 nM, and sequenced as single reads on the Illumina HiSeq 3000 (Illumina).

### ATAC-seq

Day 4 HE cells were harvested and frozen in culture media containing FBS and 5% DMSO. Cryopreserved cells were sent to Active Motif to perform the ATAC-seq assay. The cells were then thawed in a 37° C. water bath, pelleted, washed with cold PBS, and tagmented as previously described (Buenrostro et al., 2013). Briefly, cell pellets were resuspended in lysis buffer, pelleted, and tagmented using the enzyme and buffer provided in the Nextera Library Prep Kit (Illumina). Tagmented DNA was then purified using the MinElute PCR purification kit (Qiagen), amplified with 10 cycles of PCR, and purified using Agencourt AMPure SPRI beads (Beckman Coulter). Resulting material was quantified using the KAPA Library Quantification Kit for Illumina platforms (KAPA Biosystems), and sequenced with PE42 sequencing on the NextSeq 500 sequencer (Illumina).

### RNA-Seq Analysis

RNA-seq analyses were performed on three biological replicates in DOX− and DOX+ conditions. Sequencing fragments were aligned by STAR (version 2.5.2b) to human genome (hg38) with gene annotations from GENCODE (version 27). Transcript expression levels were quantified by RSEM (version 1.3.0) and differentially expression analysis was performed by DESeq2 (version 1.22.2). KEGG gene sets were defined by MSigDB (version 6.1).

### ATAC-Seq Analysis

ATAC-seq analyses were performed on two biological replicates in DOX− and DOX+ conditions. Sequencing fragments were pre-processed by the company Active Motif, Inc. Briefly, ATAC-seq reads were mapped to the human genome by BWA with default settings. Only reads that passed Illumina's purity filter, aligned with no more than 2 mismatches, and mapped uniquely to human genome were used in the subsequent analysis. Duplicate reads (“PCR duplicates”) were removed. To calculate signals, human genome was divided into 32 bp bins and the number of reads in each bin was counted. In order to smooth the data, reads were extended to 200 bp. To normalize signals across ATAC-seq datasets, the number of reads in each dataset was reduced by random sampling to the smallest number of reads present in the datasets.

ATAC-seq peaks were called by Active Motif, Inc using MACS2. For DOX− or DOX+ condition, we defined condition-specific peaks by selecting those existing in both ATAC-seq replicates of that condition and not overlapping with any peak from the two replicates of the other condition. From condition-specific peaks, we identified ‘promoter peaks’ by choosing those overlapped with protein-coding transcript's 5 kb upstream region and do not overlap with any intron or exon. DNA sequences for the 250 bp flanking regions to the center of promoter peaks were prepared for motif enrichment analysis. Motif enrichment was performed by MEME (version 5.0.4)'s CentriMo function with default settings based on motifs from HOCOMOCO human database (version 11).

ATAC-seq signals were calculated for gene's promoter region, which was defined as the 5 kb region upstream of its transcription start site (TSS). If a gene encoded for multiple transcripts, the most upstream TSS will be used as this gene's TSS. A gene's ATAC-seq promoter signal change upon DOX+ activation was computed by taking the difference of averaged signals from the two ATAC-seq replicates under DOX− or DOX+ condition. The top 5% genes that have the largest increase of ATAC-seq promoter signals were collected for GO term analysis by the Bioconductor package limma's function goana. P-values were adjusted by Benjamini & Hochberg method.

### ChIP-seq Analysis

SOX17 and IgG control ChIP-seq fragments from were aligned by BWA (version 0.7.15) with a quality threshold at 5 for read trimming and all the other options in default settings. Normalized SOX17 ChIP-seq signals were calculated by MACS2 by using all the tags at the same loci. SOX17's fold enrichment over IgG control were calculated by MACS2 using all default options.

### Statistical Analysis

Experiments were analyzed using GraphPad Prism versions 8 (GraphPad Software Inc.) and Microsoft Excel (Microsoft Corporation). Tests for statistical significance are listed with each experiment and included two-sided Student's t-test for paired analyses and one-way ANOVA, and two-way ANOVA for experiments with multiple comparisons of or grouped variables, accompanied by Tukey and Sidak post hoc tests indicated as appropriate by the software. *p<0.05, **p<0.01, ***p<0.001, and ****p<0.0001. All error bars represent the mean±SD and duplicated or triplicated independent experiments.

### Results:

SOX17 Knockout Impairs AHE Specification and Definitive Lympho-Myeloid Hematopoiesis from hPSCs

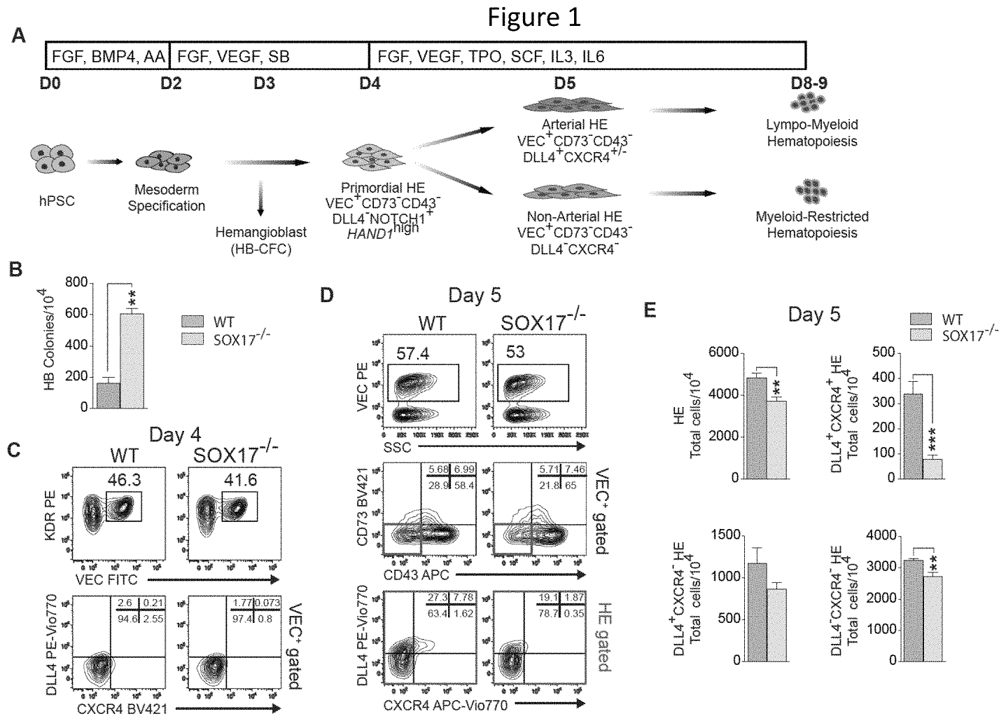

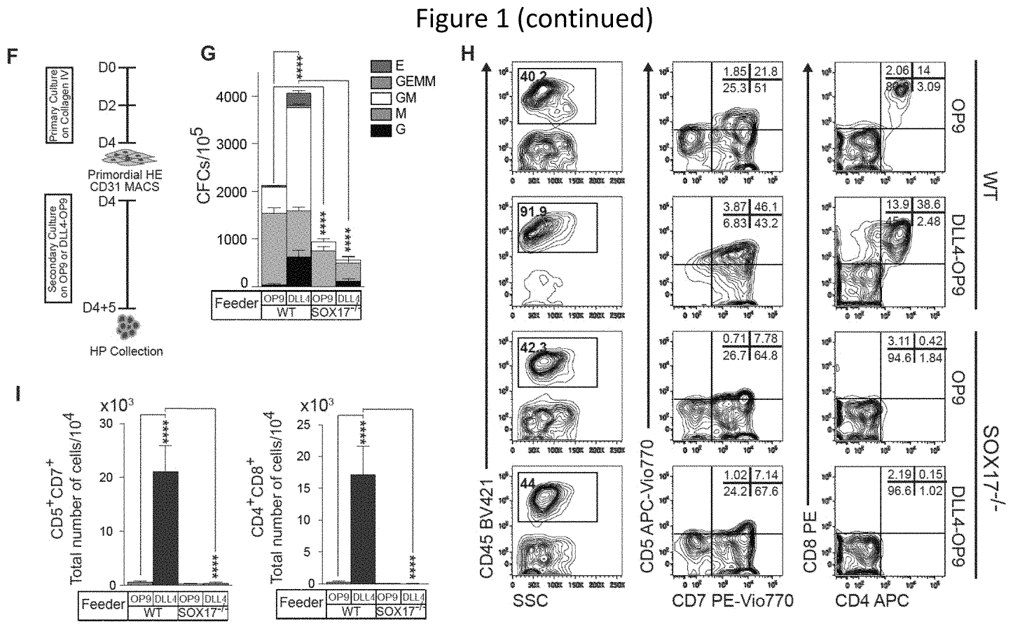

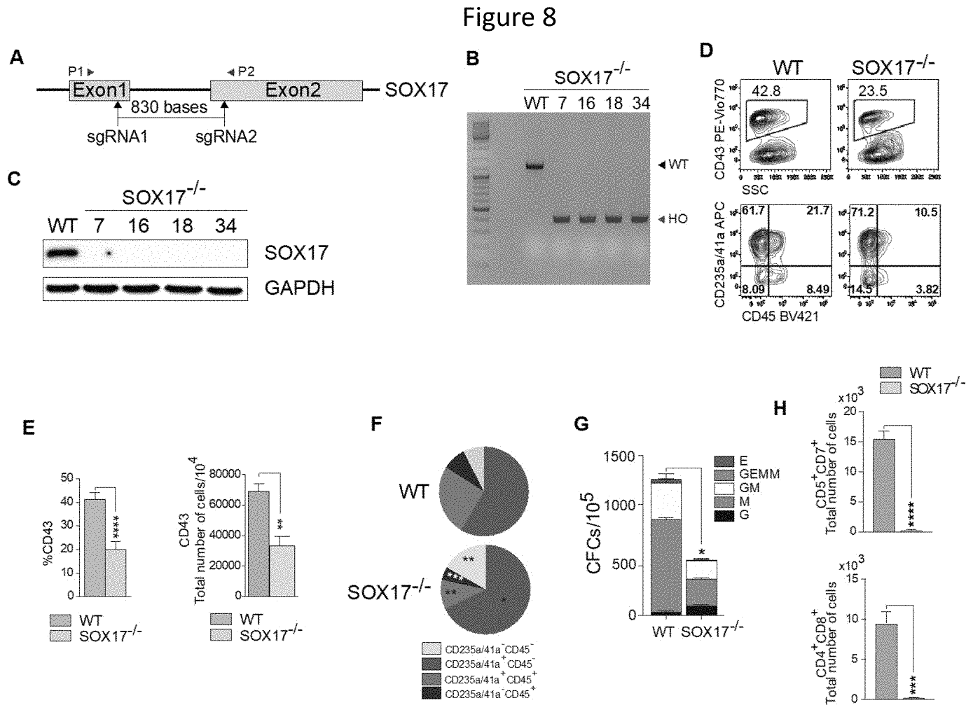

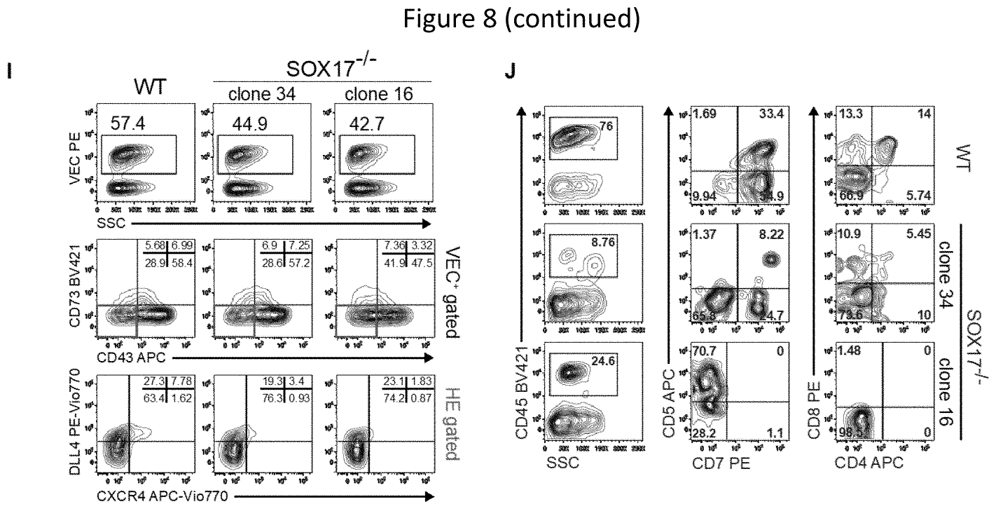

To assess the effect of SOX17 on hematopoietic development, we generated SOX17 knockout H9 human embryonic stem cell (hESC; SOX17^{-/-}) lines using CRISPR/Cas9 (FIG. 8A-C) and differentiated them into endothelial and hematopoietic cells in a chemically defined culture system (Uenishi et al., 2014). In this differentiation system, the primitive hematopoietic progenitors (HPs) with FGF2-dependent hemangioblast colony-forming cell (HB-CFCs) potential are detected on day 3 (D3) of differentiation (Choi et al., 2012; Uenishi et al., 2014; Vodyanik et al., 2010). The first immature/primordial VEC⁺CD43⁻CD73⁻NOTCH1⁺ HE cells expressing high levels of HAND1 mesodermal gene arise on D4 (D4 HE) and subsequently specify into DLL4⁺CXCR4^{+/-} arterial-type HE (AHE) with definitive lympho-myeloid potential and DLL4⁻ non-arterial-type HE (non-AHE) with myeloid-restricted potential on D5 (FIG. 1A) (Choi et al., 2012; Park et al., 2018b; Uenishi et al., 2014; Uenishi et al., 2018). As shown in FIG. 1B, SOX17 knockout significantly increased frequencies of HB-CFCs on D3, suggesting that SOX17 deficiency promotes primitive hematopoiesis. Evaluation of D4 cultures revealed that SOX17 knockout had minimal effect on D4 HE, however analysis of D5 cultures showed significantly impaired specification of DLL4⁺CXCR4⁺ AHE when compared to wild-type H9 hESCs (FIG. 1C-E).

To assess the hematopoietic potential of SOX17^{-/-} and SOX17^{+/+} HE cells and their dependence on NOTCH signaling, we isolated D4 HE cells and co-cultured them on OP9 or OP9-DLL4 (FIG. 1F.). We found that SOX17^{-/-} D4 HE had significantly reduced myeloid (FIG. 1G) and no T lymphoid potential (FIG. 1H-I). Importantly, there was no change in hematopoietic potential of D4 SOX17^{-/-} HE between coculture on OP9 and OP9-DLL4 (FIG. 1G), indicating that SOX17^{-/-} cells lack NOTCH-dependent hematopoietic potential. Similarly, hematopoietic cells collected on D9 from SOX17^{-/-} hESCs demonstrated decreased myeloid CFC and T lymphoid potential along with a decreased CD43⁺ cells and proportion of CD45⁺ cells within the CD43⁺ population, as compared to wild-type cells (FIG. 8D-H). These effects on HE and T cells from SOX17 deficiency were confirmed using two different SOX17 knockout clones (FIG. 8I-J). The lack of NOTCH sensitivity and abrogation of T lymphoid potential suggests that SOX17^{-/-} HE cells do not possess definitive lympho-myeloid potential (Hadland et al., 2004; Kennedy et al., 2012; Kumano et al., 2003; Robert-Moreno et al., 2005).

Overall, these results demonstrate the critical role of SOX17 in the specification of definitive lympho-myeloid hematopoiesis and DLL4⁺CXCR4⁺ AHE from hPSCs. Although both, DLL4 and CXCR4, are considered markers of AHE (Chong et al., 2011; Yamamizu et al., 2010), DLL4 expression has been found in arterial vessels of yolk sac and aorta (Duarte et al., 2004; Herman et al., 2018; Robert-Moreno et al., 2005), while CXCR4 expression was detected in aorta and vitelline/umbilical arteries (McGrath et al., 1999; Werner et al., 2020), i.e. vasculature harboring precursors capable of maturing into definitive HSCs (Dzierzak and Medvinsky, 1995; Gordon-Keylock et al., 2013), but not in yolk sac (McGrath et al., 1999; Venkatesh et al., 2008; Werner et al., 2020). Thus, SOX17 is the most essential factor for the formation of AHE with the CXCR4⁺ phenotype typical of HE with HSC potential in vivo.

### SOX17 Induction at the Mesodermal Stage Enhances AHE Specification and Definitive Lympho-Myeloid Hematopoiesis

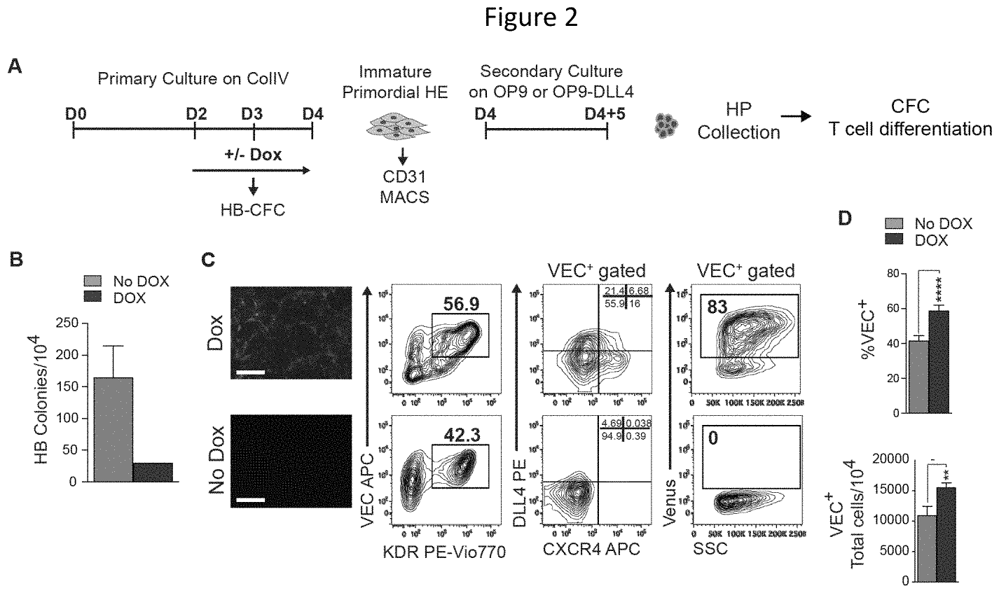

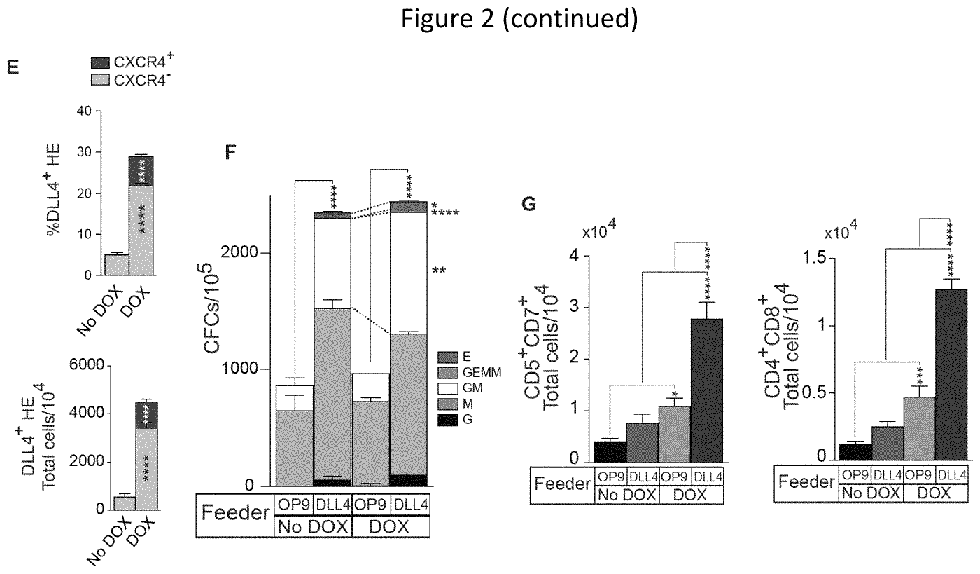

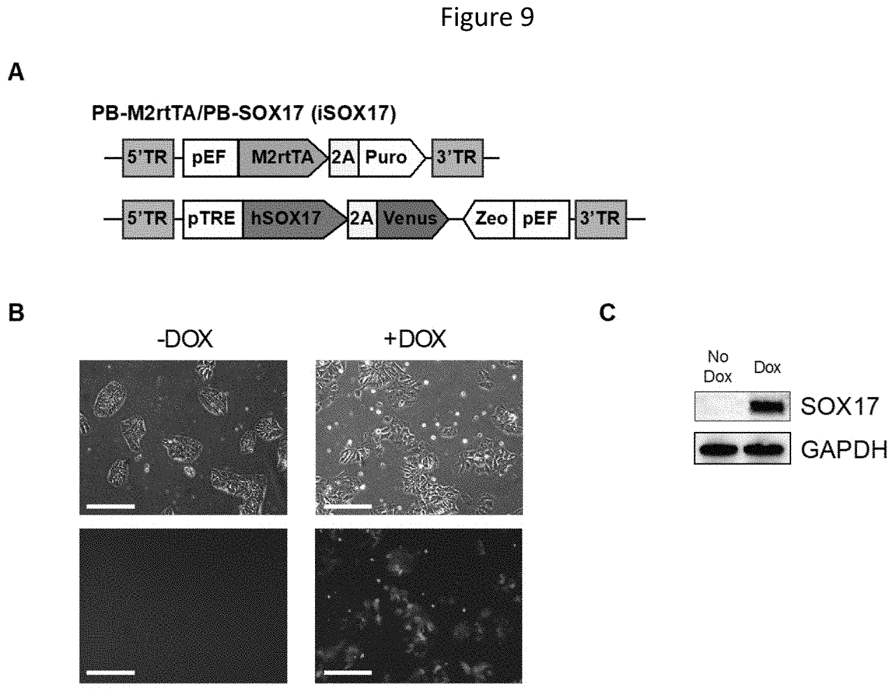

To further characterize the role of SOX17 during hematoendothelial development, we engineered an H9 hESC line with a transgene cassette that expresses SOX17 upon treatment with doxycycline (DOX; iSOX17-hESCs; FIG. 9). We then differentiated inducible SOX17 (iSOX17) cells with or without DOX, starting from the mesodermal stage of development (D2) through D4, to analyze the effect on specification of HB-CFCs (D3) and D4 HE specification (FIG. 2A). As shown in FIG. 2B, SOX17 induction significantly reduced the numbers of primitive HPs on D3, as evidenced by the decrease in HB-CFCs. This observation is consistent with murine studies that demonstrated suppression of primitive hematopoiesis by Sox17 (Serrano et al., 2010). Concordantly, SOX17 induction increased production of VEC⁺ HE on D4, along with early upregulation of DLL4 and CXCR4 when HE cells are typically DLL4⁻CXCR4⁻ (Park et al., 2018b; Uenishi et al., 2018), as seen in No DOX cultures (FIG. 2C). Assessment of hematopoietic potential from D4 HE revealed that blood cells collected from DOX+ conditions had higher T cell potentials from both HE/OP9 and HE/OP9-DLL4 cocultures, as compared to D4 HE collected from DOX− conditions (FIG. 2F-G). In addition, D4 HE from DOX+ cultures demonstrated more robust multipotent myeloid and lymphoid hematopoiesis following exposure to DLL4 NOTCH ligand (OP9 vs OP9-DLL4 hematopoiesis), while these OP9 vs OP9-DLL4 differences in DOX− D4 HE cocultures were more modest.

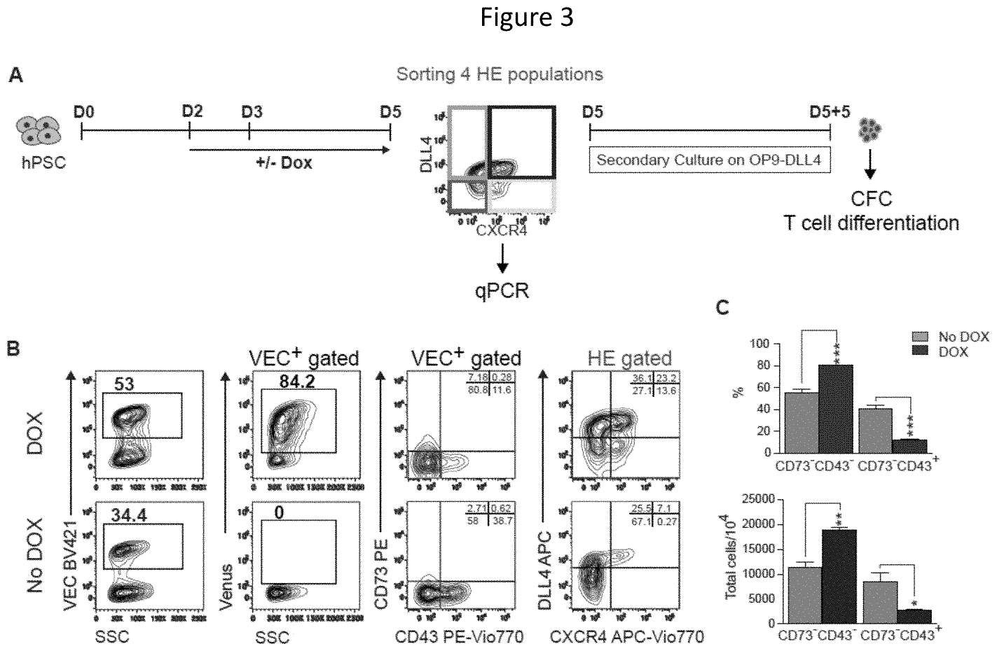

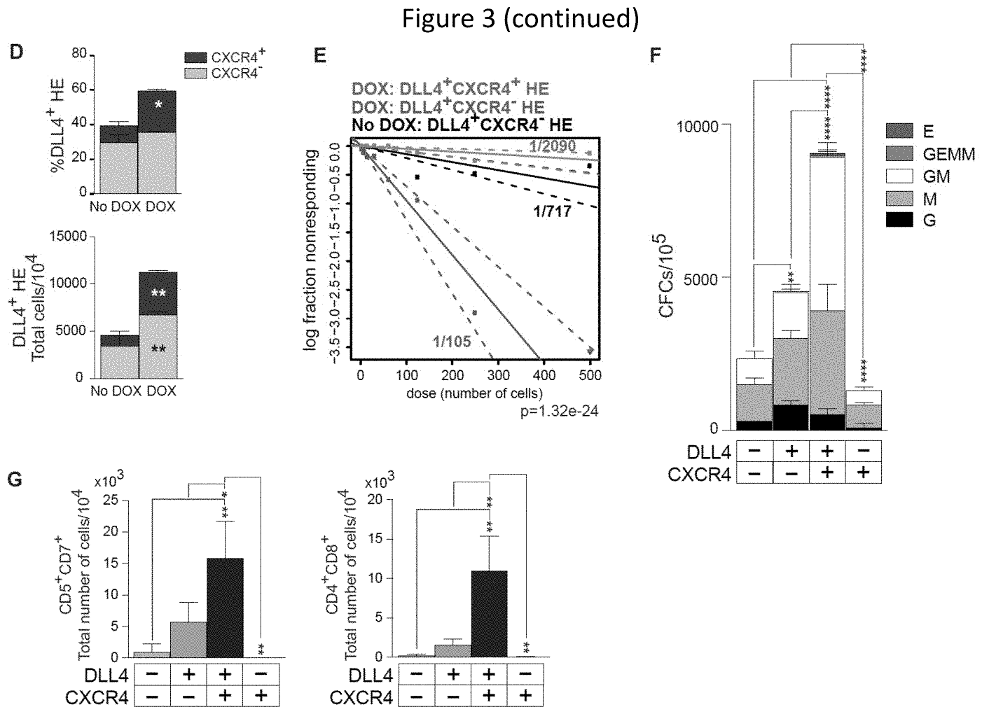

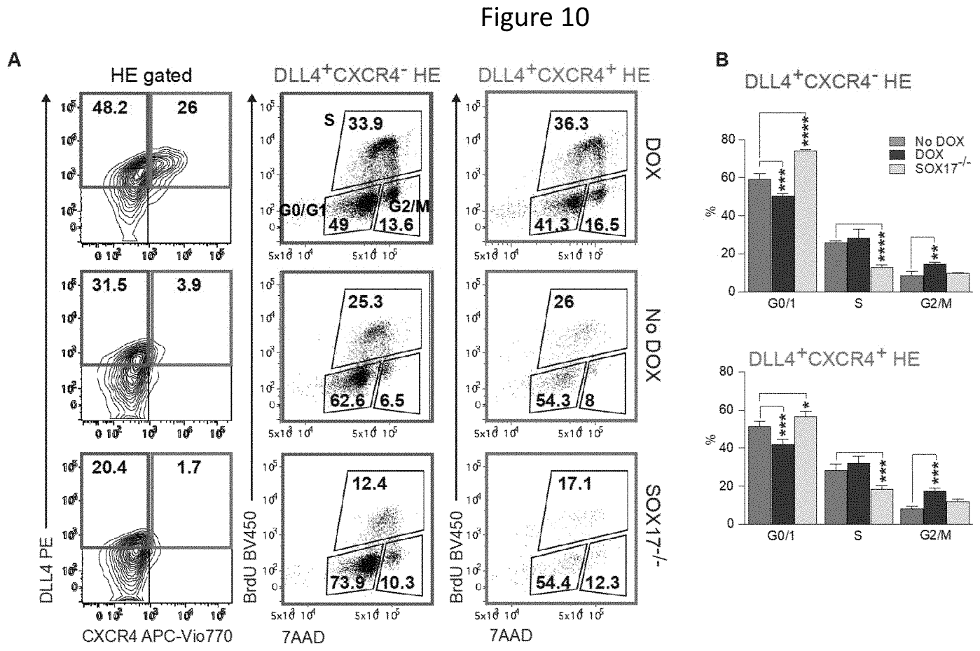

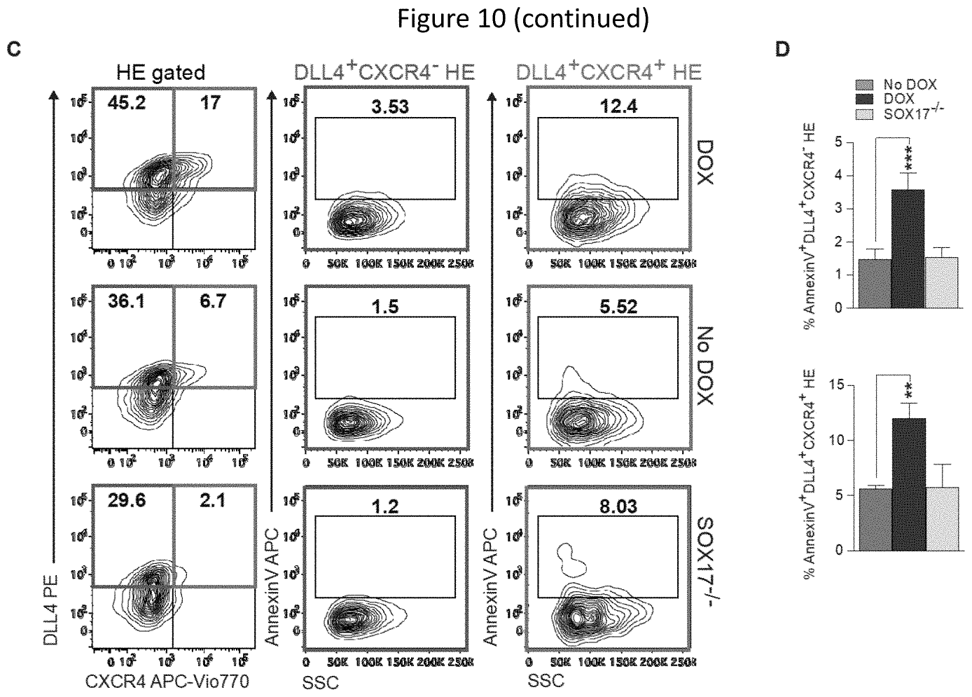

As we previously demonstrated, D4 HE specifies 24 hours later (D5) into DLL4⁺CXCR4^{+/-} arterial HE and non-arterial DLL4⁻CXCR4⁻ HE (Park et al., 2018b; Uenishi et al., 2018). Assessment of HE phenotype on D5 (FIG. 3A) reveals that SOX17 upregulation significantly increased the percentage of VECCD73⁻CD43⁻ HE cells, and expression of arterial markers, DLL4 and CXCR4, within this population, predominantly increasing the DLL4⁺CXCR4⁺ HE population (FIG. 3B-D). In addition, we note that DOX treatment also induced the DLL4⁻CXCR4⁺ subpopulation within phenotypical (VECCD73⁻CD43⁻) HE subset (FIG. 3B). Studies of SOX17 in mice have shown that this factor controls proliferation of hematopoietic and endothelial cells (Clarke et al., 2013; Kim et al., 2007; Liu et al., 2019; Nobuhisa et al., 2014; Serrano et al., 2010). Examination of the proliferative potential of HE subsets using BrdU and cell cycle analysis, revealed that SOX17 overexpression led to a significant cell cycle shift from GO/G1 to S and G2/M phases in CXCR4⁻ and CXCR4⁺ AHE subsets (FIG. 10A-B). In contrast, a significant decrease in cycling cells was observed in SOX17^{-/-} DLL4⁺ populations. Although similar to mouse studies (Serrano et al., 2010), we found that DOX treatment increased apoptosis along with increased proliferation of AHE (FIG. 10C-D), the total output of viable AHE cells in DOX+ conditions was significantly higher as compared to DOX− conditions (FIG. 3C-D). Limiting dilution analysis of the hematopoietic potential of AHE subsets revealed that following DOX treatment, the frequency of hemogenic cells was the highest in DLL4⁺CXCR4⁻ population while their frequency in the DLL4⁺CXCR4⁺ subset was 20-fold lower. In addition, we found that DOX treatment increased the frequency of hemogenic cells in DLL4⁺CXCR4⁻ population by nearly 7-fold as compared to DOX− conditions (FIG. 3E). To assess the hemogenic potential of four D5 HE subsets (DLL4⁺CXCR4⁺, DLL4⁺CXCR4⁻, DLL4⁻CXCR⁻ DLL4⁻CXCR4⁺), each subpopulation was isolated by FACS cell sorting and analyzed for CFC and T cell potential following culture on OP9-DLL4 (FIG. 3A). In our prior studies, we found that coculture with OP9-DLL4 is essential to induce EHT from AHE (Uenishi et al., 2018). Consistent with our prior observations (Park et al., 2018b), the DLL4⁺CXCR4⁺ HE subset had the most robust CFC and T cell potentials (FIG. 3F-G), despite having the lowest frequency of hemogenic cells as found in the limiting dilution assay (FIG. 3E). In contrast, CXCR4⁺DLL4⁻ HE cells possessed very limited myeloid potential and were completely devoid of T cell potential. Thus, we concluded that SOX17-induced CXCR4⁺DLL4⁻ cells are mostly nonhemogenic or primitive hemogenic progenitors and excluded them from further analysis.

Collectively, these studies suggest that SOX17 upregulation promotes definitive lympho-myeloid hematopoiesis from hPSCs through enhancement of AHE specification with CXCR4⁺DLL4⁺ phenotype, typical for HE at sites of HSC emergence.

### SOX17 Overexpression Activates NOTCH-Mediated Arterial Program and Expression of HOXA Cluster Genes in HE

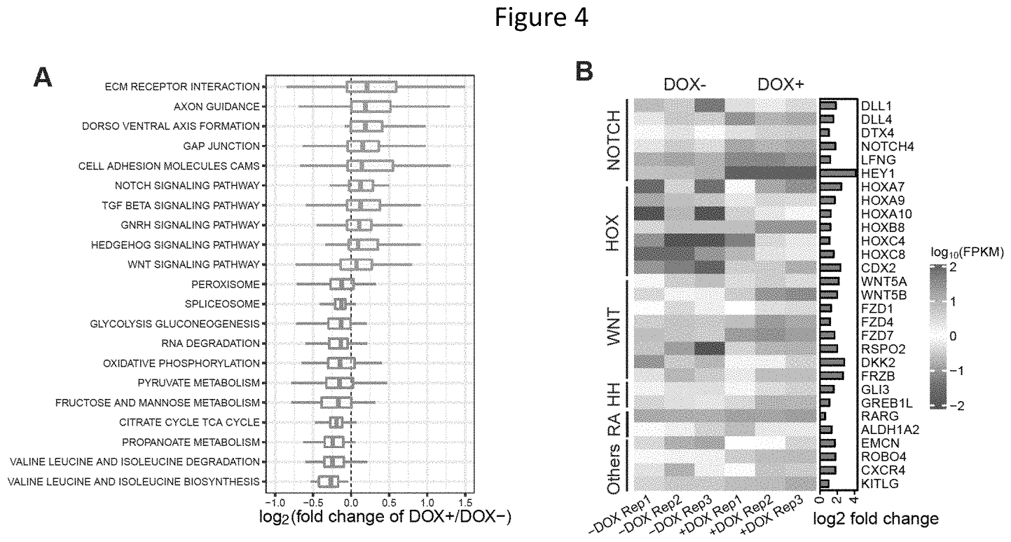

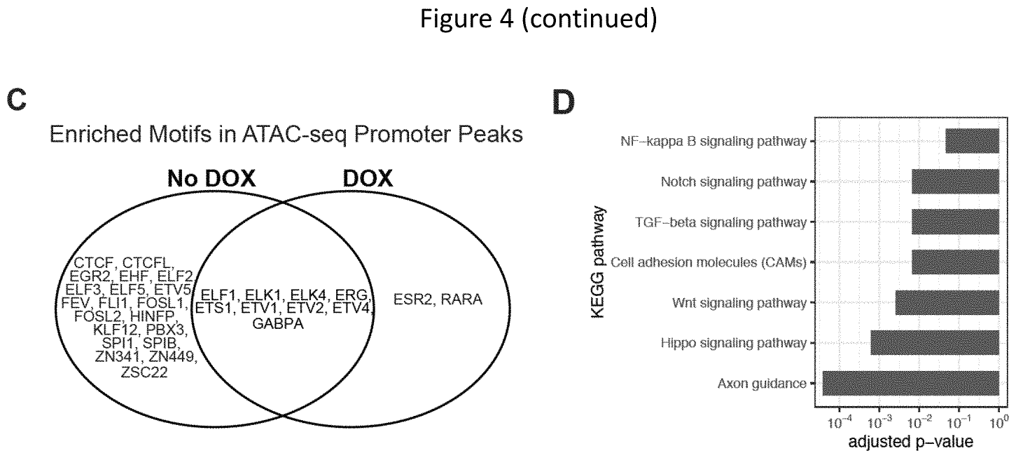

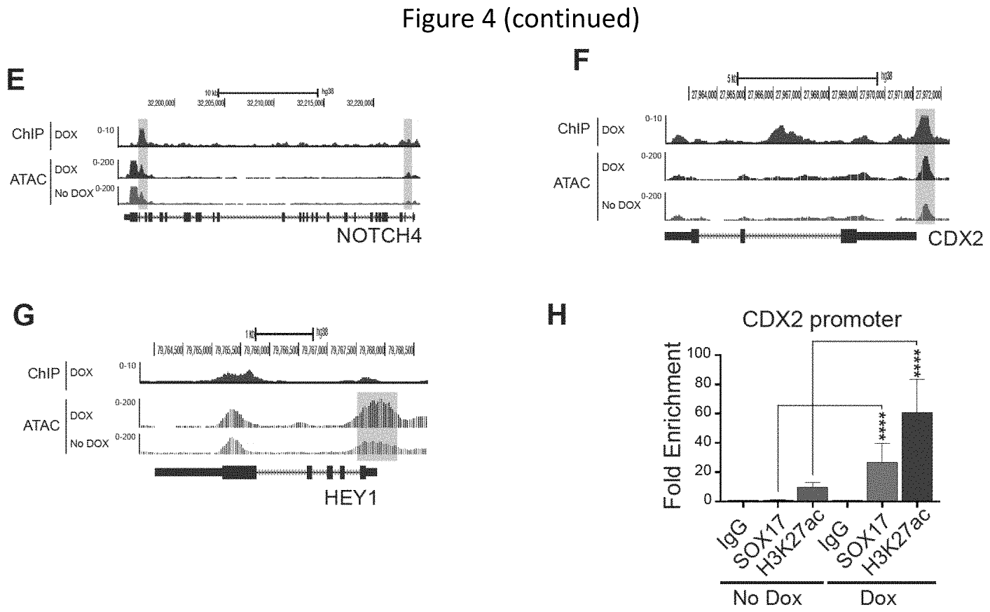

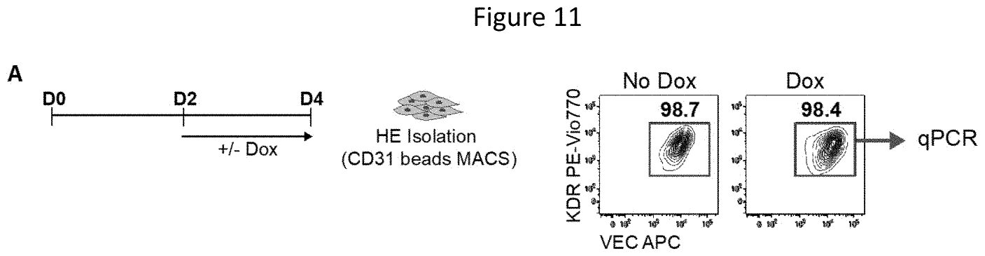

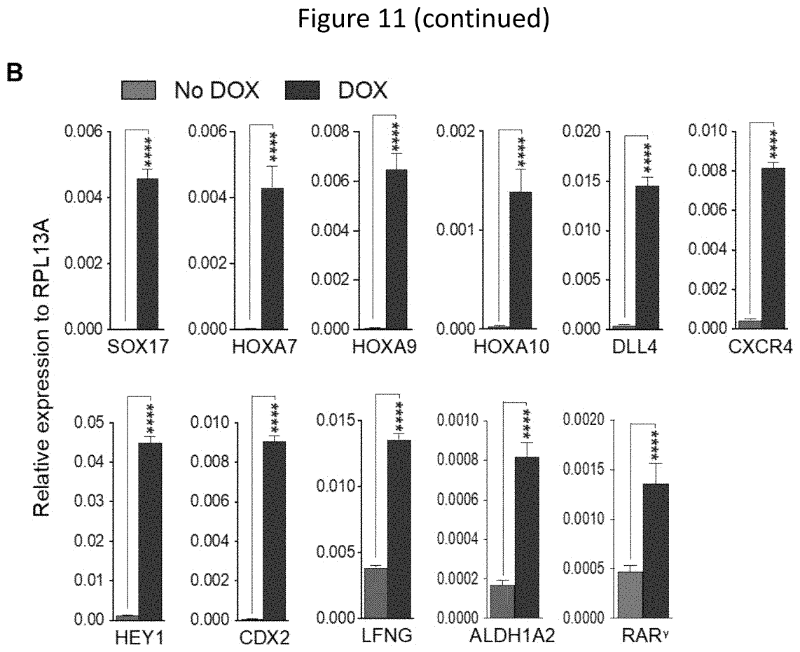

To understand the molecular mechanisms of the effect of SOX17, we performed molecular profiling of D4 HE from DOX+ and DOX− cultures using RNA-seq and ATAC-seq. To analyze chromatin binding of SOX17 by ChIP-seq, we used DOX+ cultures because under DOX− conditions, SOX17 expression was absent in D4 HE, i.e., before AHE was formed (Uenishi et al., 2018) (FIG. 11). Gene set enrichment analysis (GSEA) of RNA-seq data revealed enrichment in KEGG categories related to dorso-ventral axis formation: NOTCH, TGFβ, Hedgehog and WNT signaling pathways with downregulation of multiple metabolic pathways in DOX+ as compared to DOX− (FIG. 4A and Table 3). The upregulated genes included HOX genes, HOXA genes (HOXA7, HOXA9, HOXA10) and CDX2, as well as key molecules in NOTCH (DLL1, DLL4, NOTCH4) and WNT signaling (WNT5A, WNT5B). In addition, we noted upregulation of molecules associated with retinoic acid signaling (ALDH1A2 and RARG) and molecules associated with HSC development (EMCN, ROBO4, KITLG) (FIG. 4B). Upregulation of these genes was confirmed by qPCR (FIG. 11).

TABLE 3
Enriched KEGG terms from day 4 HE RNAseq

| Gene set name | Up-regulated in | Adjusted p-value |
|---|---|---|
| KEGG_AXON_GUIDANCE | DOX+ | 2.10837179648431e−8 |
| KEGG_CELL_ADHE_SION_MOLECULES_CAMS | DOX+ | 2.10837179648431e−8 |
| KEGG_GLYCOSAMINOGLYCAN_BIOSYNTHESIS_CHONDROITIN_SULFATE | DOX+ | 2.10837179648431e−8 |
| KEGG_RIBOSOME | DOX− | 6.73770384348161e−8 |
| KEGG_ECM_RECEPTOR_INTERACTION | DOX+ | 1.00763E−06 |
| KEGG_BASAL_CELL_CARCINOMA | DOX+ | 4.48645E−06 |
| KEGG_GLYCO_SAMINOGLYCAN_BIOSYNTHESIS_HEPARAN_SULFATE | DOX+ | 5.33193E−05 |
| KEGG_HEDGEHOG_SIGNALING_PATHWAY | DOX+ | 0.000133684 |
| KEGG_AMINOACYL_TRNA_BIOSYNTHESIS | DOX− | 0.000276991 |
| KEGG_FOCAL_ADHESION | DOX+ | 0.00051598 |
| KEGG_VALINE_LEUCINE_AND_ISOLEUCINE_DEGRADATION | DOX− | 0.001401729 |
| KEGG_PROPANOAIL_METABOLISM | DOX− | 0.002156859 |
| KEGG_MELANOGENESIS | DOX+ | 0.002156859 |
| KEGG_GAPJUNCTION | DOX+ | 0.002241927 |
| KEGG_GLYCO_SPHINGOLIPID_BIOSYNTHESIS_LACTO_AND_NEOLACTO_SERIES | DOX+ | 0.00238555 |
| KEGG_OXIDATIVE_PHOSPHORYLATION | DOX− | 0.003148941 |
| KEGG_ARRHYTHMOGENIC_RIGHT_VENTRICULAR_CARDIOMYOPATHY_ARVC | DOX+ | 0.003281063 |
| KEGG_RNA_DEGRADATION | DOX− | 0.003281063 |
| KEGG_TGF_BETA_SIGNALING_PATHWAY | DOX+ | 0.003288519 |
| KEGG_SPLICEOSOME | DOX− | 0.003288519 |
| KEGG_MELANOMA | DOX+ | 0.003288519 |
| KEGG_PATHWAYS_IN_CANCER | DOX+ | 0.00460277 |
| KEGG_RNA_POLYMERASE | DOX− | 0.00460277 |
| KEGG_PEROXISOME | DOX− | 0.006024659 |
| KEGG_PYRUVAIL_METABOLISM | DOX− | 0.006685177 |
| KEGG_FRUCTOSE_AND_MANNOSE_METABOLISM | DOX− | 0.008549323 |
| KEGG_GLYCOLYSIS_GLUCONEOGENESIS | DOX− | 0.009118394 |
| KEGG_PROSTATE_CANCER | DOX+ | 0.010329457 |
| KEGG_NOTCH_SIGNALING_PATHWAY | DOX+ | 0.010329457 |
| KEGG_ONE_CARBON_POOL_BY_FOLATE | DOX− | 0.011068986 |
| KEGG_HUNTINGTONS_DISEASE | DOX− | 0.011068986 |
| KEGG_DORSO_VENTRAL_AXIS_FORMATION | DOX+ | 0.011068986 |
| KEGG_DILATED_CARDIOMYOPATHY | DOX+ | 0.011676678 |
| KEGG_CITRATE_CYCLEJCA_CYCLE | DOX− | 0.012849034 |
| KEGG_VALINE_LEUCINE_AND_ISOLEUCINE_BIOSYNTHESIS | DOX− | 0.027921813 |
| KEGG_ENDOMETRIAL_CANCER | DOX+ | 0.031692773 |
| KEGG_GNRH_SIGNALING_PATHWAY | DOX+ | 0.037610248 |
| KEGG_WNT_SIGNALING_PATHWAY | DOX+ | 0.038958319 |
| KEGG_GLYCO_SPHINGOLIPID_BIOSYNTHESIS_GANGLIO_SERIES | DOX+ | 0.039893448 |
| KEGG_RENAL_CELL_CARCINOMA | DOX+ | 0.047099844 |

ATAC-seq analysis of D4 HE isolated from DOX-treated cultures identified 93,615 and 100,036 open chromatin regions in the two ATAC-seq replicates, respectively, of which 5,130 of which were specific to DOX+ conditions. Gene Ontology (GO) analysis of genes with increased ATAC-seq counts at promoters upon DOX treatment revealed enrichment in categories associated with development and morphogenesis, including blood vessel morphogenesis (Table 4), suggesting that SOX17 facilitates the establishment of gene regulatory networks essential for early morphogenesis, including vascular development. Motif-enrichment analysis of ATAC-seq peaks at promoters in DOX+ and DOX− conditions revealed enrichment in ETS-binding motifs for both conditions, consistent with the endothelial nature of the analyzed cells. However, in DOX+ conditions we observed a unique enrichment in retinoic receptor alpha (RARA) and estrogen receptor 2 (ESR2) motifs at open chromatin regions (FIG. 4C, Table 5).

TABLE 4
Top 5% of genes with ATAC-seq signals increased
at promoters upon DOX induction

| GO term | Adjusted p-value |
|---|---|
| anatomical structure morphogenesis | 1.78003407755937e−7 |
| nervous system development | 2.85807041113842e−7 |
| neurogenesis | 4.58194E−06 |
| central nervous system development | 8.16944E−06 |
| tube morphogenesis | 2.44821E−05 |
| system development | 2.44821E−05 |
| generation of neurons | 2.92474E−05 |
| tube development | 0.000110693 |
| multicellular organism development | 0.000110693 |
| regulation of nervous system development | 0.000161455 |
| regulation of multicellular organismal development | 0.000161455 |
| anatomical structure development | 0.000185597 |
| neuron differentiation | 0.000185597 |
| locomotion | 0.000185597 |
| regulation of anatomical structure morphogenesis | 0.000185597 |
| cell migration | 0.000246498 |
| regulation of developmental process | 0.000342946 |
| head development | 0.000342946 |
| positive regulation of developmental process | 0.000465449 |
| blood vessel morphogenesis | 0.000758105 |
| neuron development | 0.000758105 |
| adenylate cyclase-modulating G protein-coupled | 0.000761229 |
| receptor signaling pathway | |
| neuron projection development | 0.000761229 |
| vasculature development | 0.000865103 |
| anatomical structure formation involved in | 0.000920372 |
| morphogenesis | |
| neuron part | 0.001038012 |
| developmental process | 0.001038012 |
| angiogenesis | 0.00105143 |
| cardiovascular system development | 0.00105143 |
| embryonic morphogenesis | 0.001067191 |
| basement membrane | 0.001075712 |
| brain development | 0.001301484 |
| cellular developmental process | 0.001476637 |
| animal organ morphogenesis | 0.001521223 |
| neuron projection | 0.001521223 |
| cell motility | 0.001521223 |
| localization of cell | 0.001521223 |
| blood vessel development | 0.001567922 |
| intrinsic component of plasma membrane | 0.001593111 |
| regulation of cell development | 0.001593111 |
| regulation of neurogenesis | 0.001610939 |
| animal organ development | 0.001714315 |
| multicellular organismal process | 0.001735658 |
| G protein-coupled receptor signaling pathway, | 0.002324971 |
| coupled to cyclic nucleotide second messenger | |
| integral component of plasma membrane | 0.002408441 |
| circulatory system development | 0.002859264 |
| regulation of multicellular organismal process | 0.003115049 |
| cell development | 0.003234361 |
| axon development | 0.003618464 |
| cell differentiation | 0.003626835 |
| embryonic organ development | 0.003626835 |
| cell part morphogenesis | 0.006182665 |
| embryo development | 0.007461113 |
| signaling receptor binding | 0.008843617 |
| synapse | 0.008843617 |
| movement of cell or subcellular component | 0.009048212 |
| cellular component morphogenesis | 0.009048212 |
| tumor necrosis factor-activated receptor activity | 0.009048212 |
| renal system development | 0.009737108 |
| tissue morphogenesis | 0.009737108 |
| axonogenesis | 0.01004697 |
| regulation of neuron differentiation | 0.010178993 |
| positive regulation of cell development | 0.010271038 |
| axon | 0.010271038 |
| adenylate cyclase-inhibiting G protein-coupled | 0.010271038 |
| receptor signaling pathway | |
| regulation of cell differentiation | 0.011531827 |
| positive regulation of nervous system development | 0.012459588 |
| ear development | 0.012623805 |
| positive regulation of cell differentiation | 0.013451981 |
| metanephros morphogenesis | 0.015724879 |
| synaptic membrane adhesion | 0.015740799 |
| ear morphogenesis | 0.015896 |
| inner ear morphogenesis | 0.016315176 |
| cell morphogenesis involved in neuron | 0.017844159 |
| differentiation | |
| neuron projection morphogenesis | 0.020119928 |
| chondrocyte differentiation | 0.020119928 |
| plasma membrane bounded cell projection | 0.020119928 |
| morphogenesis | |
| positive regulation of epithelial cell migration | 0.020320479 |
| system process | 0.020320479 |
| cell morphogenesis | 0.020320479 |
| collagen-containing extracellular matrix | 0.020320479 |
| inner ear development | 0.02054135 |
| positive regulation of protein phosphorylation | 0.02187698 |
| cell projection morphogenesis | 0.02187698 |
| positive regulation of multicellular organismal | 0.02187698 |
| process | |
| second-messenger-mediated signaling | 0.02187698 |
| sensory organ development | 0.022685916 |
| positive regulation of neurogenesis | 0.022685916 |
| nephron development | 0.023439655 |
| chemotaxis | 0.023462666 |
| death receptor activity | 0.023660162 |
| regulation of system process | 0.024178531 |
| cAMP-mediated signaling | 0.024615989 |
| taxis | 0.024615989 |
| positive regulation of cell proliferation | 0.0260098 |
| sensory organ morphogenesis | 0.0260098 |
| cell proliferation | 0.028080528 |
| positive regulation of cell migration | 0.029165326 |
| negative regulation of apoptotic process | 0.032275505 |
| cell morphogenesis involved in differentiation | 0.03348346 |
| growth factor activity | 0.03348346 |
| extracellular matrix | 0.033910658 |
| dichotomous subdivision of terminal units | 0.035191974 |
| involved in lung branching | |
| extracellular matrix organization | 0.036705001 |
| extracellular structure organization | 0.036705001 |
| intrinsic component of membrane | 0.036875421 |
| cell communication | 0.038652168 |
| kidney morphogenesis | 0.039463928 |
| dopamine transport | 0.040553679 |
| regulation of neuron projection development | 0.041435175 |
| positive regulation of locomotion | 0.042212449 |
| metal ion transmembrane transporter activity | 0.042443184 |
| regulation of developmental growth | 0.042443184 |
| urogenital system development | 0.042443184 |
| regulation of cell migration | 0.044587154 |
| regulation of cell proliferation | 0.045698414 |
| positive regulation of phosphorylation | 0.048504286 |
| receptor tyrosine kinase binding | 0.049864381 |

TABLE 5
Enriched motifs in condition-specific ATAC-seq promoter peaks

| Motif | Condition | Adjusted p-value |
|---|---|---|
| ETS1 | DOX+ | 0.0000032 |
| ETV1 | DOX+ | 0.00022 |
| ELK4 | DOX+ | 0.00023 |
| ETV2 | DOX+ | 0.00043 |
| ELF1 | DOX+ | 0.0009 |
| ELK1 | DOX+ | 0.002 |
| ERG | DOX+ | 0.0026 |
| GABPA | DOX+ | 0.0047 |
| ESR2 | DOX+ | 0.012 |
| ETV4 | DOX+ | 0.018 |
| RARA | DOX+ | 0.025 |
| GABPA | DOX− | 1.4e−13 |
| ELF1 | DOX− | 1.9e−13 |
| ETV1 | DOX− | 9.6e−13 |
| ELF2 | DOX− | 1.2e−12 |
| ELF5 | DOX− | 2.7e−10 |
| ETS1 | DOX− | 2.2e−9 |
| ETV2 | DOX− | 2.6e−9 |
| ERG | DOX− | 1.5e−8 |
| ELK4 | DOX− | 3.2e−8 |
| ETV4 | DOX− | 9.8e−8 |
| ELK1 | DOX− | 5.6e−7 |
| EHF | DOX− | 9.2e−7 |
| ELF3 | DOX− | 0.0000071 |
| SPI1 | DOX− | 0.000012 |
| FOSL1 | DOX− | 0.00011 |
| ETV5 | DOX− | 0.0002 |
| CTCF | DOX− | 0.00025 |
| ZN341 | DOX− | 0.00045 |
| FLI1 | DOX− | 0.00061 |
| SPIB | DOX− | 0.0011 |
| ZSC22 | DOX− | 0.0014 |
| FEV | DOX− | 0.0019 |
| ZN449 | DOX− | 0.0026 |
| CTCFL | DOX− | 0.0034 |
| FOSL2 | DOX− | 0.0071 |
| PBX3 | DOX− | 0.0077 |
| EGR2 | DOX− | 0.015 |
| HINFP | DOX− | 0.019 |
| KLF12 | DOX− | 0.023 |

To identify direct targets of SOX17 in D4 HE, we analyzed overlapping SOX17 ChIP-seq and ATAC-seq peaks at promoters and intragenic regions of differentially expressed genes (DEGs) between DOX+ and DOX− conditions. The set of DEGs bound by SOX17 at open chromatin regions was enriched in the Hippo, Wnt, TGFβ, and Notch signaling pathways, and included genes important for NOTCH regulation, arterial specification and hematopoietic development such as NOTCH4, CDX2 (FIG. 4E-F, Table 6), DLL1, LFNG, DTX4, KITLG, HLX, GLI3, EOMES, DKK2, WNT5B, and PRDM16 (Table 6). Selective quantitative ChIP analysis by PCR confirmed SOX17 binding at the CDX2 promoter and demonstrated a prominent increase in levels of activating H3K27ac at this site in DOX+ cultures (FIG. 4H). However, HOXA genes, except HOXA10, were not found within this set of genes, suggesting that SOX17 regulates the HOXA cluster indirectly. Similarly, we noted a substantial increase in ATAC-seq counts at the HEY1 promoter following DOX treatment without SOX17 binding (FIG. 4G), reflecting an indirect activation of downstream NOTCH targets following SOX17 overexpression (FIG. 4F).

TABLE 6
DEGs with overlapping TS and intragenic
SOX17 and ATAC peaks

| KEGG | Adjusted p-value |
|---|---|
| Axon guidance | 0.000039 |
| Hippo signaling pathway | 0.00061 |
| Prostate cancer | 0.0025 |
| Parathyroid hormone synthesis, secretion | 0.0026 |
| and action | |
| Wnt signaling pathway | 0.0026 |
| Pathways in cancer | 0.0038 |
| EGFR tyrosine kinase inhibitor resistance | 0.0047 |
| Cell adhesion molecules (CAMs) | 0.0066 |
| TGF-beta signaling pathway | 0.0066 |
| Proteoglycans in cancer | 0.0066 |
| Breast cancer | 0.0066 |
| Gastric cancer | 0.0066 |
| Notch signaling pathway | 0.0066 |
| MicroRNAs in cancer | 0.011 |
| Hippo signaling pathway-multiple species | 0.013 |
| ECM-receptor interaction | 0.036 |
| Morphine addiction | 0.039 |
| Protein digestion and absorption | 0.042 |
| Endocrine resistance | 0.042 |
| Melanogenesis | 0.048 |
| NF-kappa B signaling pathway | 0.048 |

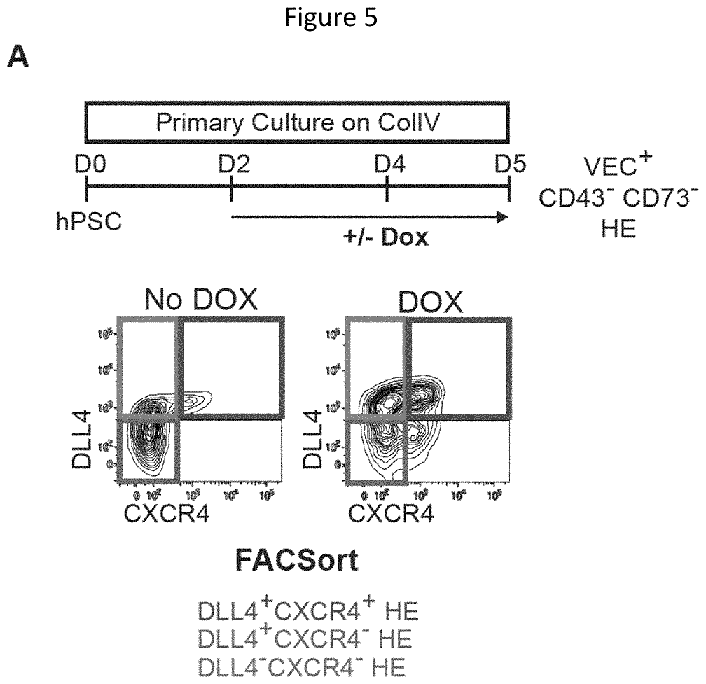

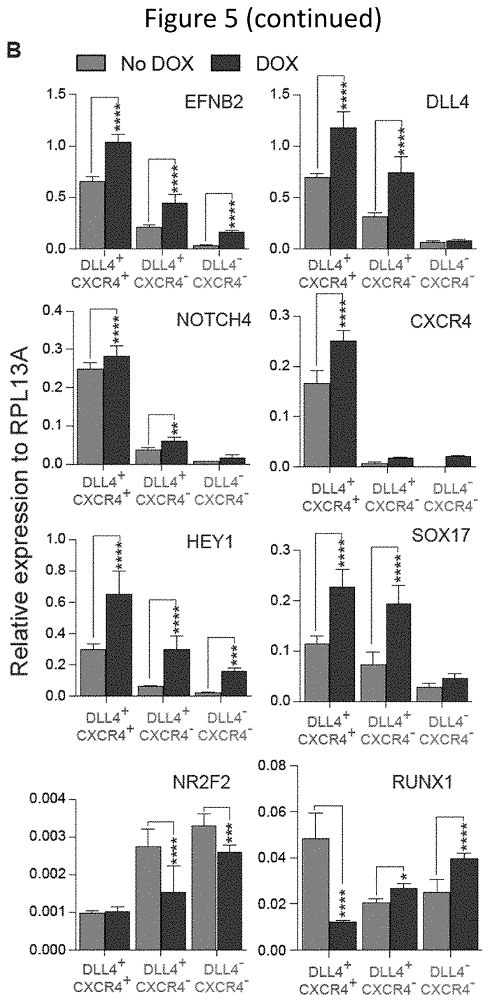

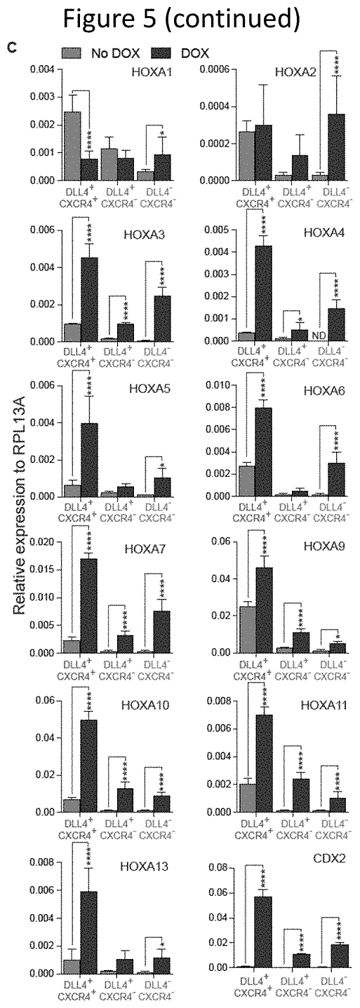

To confirm the role of SOX17 in establishing the HOXA pattern in HE, we evaluated expression of arterial and HOXA genes in the three major subsets of HE on D5 (FIG. 5A), excluding the DLL4⁻CXCR4⁺ population, which has a very limited hematopoietic potential (FIG. 3E-F). As shown in FIG. 5B, in DOX− cultures, the DLL4⁺CXCR4⁺ HE subpopulation expressed the highest levels of arterial genes, including SOX17, and lowest levels of NR2F2 venous gene as compared to two other HE subsets. SOX17 overexpression upregulated expression of arterial markers EFNB2, DLL4, NOTCH4, CXCR4 and HEY1 in all three HE subsets, with the highest levels of expression observed in DLL4⁺CXCR4⁺ HE subpopulation. Similarly, we observed significant upregulation of CDX2 and HOXA (HOXA7, HOXA9, HOXA10, HOX11) gene expression in all three subsets of HE with the highest levels of these HOXA genes observed in the DLL4⁺CXCR4⁺ HE subpopulation, while the lowest level of HOXA gene expression was observed in DLL4⁻CXCR4⁻ non-AHE (FIG. 5C). In addition, upregulation of more anterior HOXA3 through HOXA6 genes was observed in DLL4⁺CXCR4⁺ and DLL4⁻CXCR4⁻ HE, although their level of expression was much lower as compared to more distal HOXA genes. We also noted inverse correlation between levels of SOX17 and RUNX1 expression (FIG. 5B), consistent with prior findings of negative regulation of Runx1 by Sox17 in the murine embryo (Clarke et al., 2013; Lizama et al., 2015). However, suppression of RUNX1 following SOX17 upregulation was observed only in the DLL4⁺CXCR4⁺ AHE subset, while upregulation of SOX17 in DLL4⁺CXCR4⁻ subset was associated with increased RUNX1 expression suggesting stage-specific differences in RUNX1 regulation following AHE formation from hESCs.

QPCR analysis of phenotypically similar HE populations generated from SOX17^{-/-} and wild type hESCs revealed substantial reduction of arterial genes in both DLL4⁺CXCR4⁻ and DLL4⁺CXCR4⁺ AHE subsets as compared to wild type cells. However, significant reduction in HOXA5-HOX10 gene expression was observed only in DLL4⁺CXCR4⁺ AHE subset (FIG. 12A-C). This downregulation of HOXA genes in SOX17^{-/-} cells was associated with a significant reduction of CDX2 expression in AHE, while no differences in CDX2 expression were observed in non-AHE (FIG. 12C). Thus, these findings confirm the essential role of SOX17 in arterial specification of HE and establishing HOXA signature in DLL4⁺CXCR4⁺ AHE.

Taken together, our molecular profiling studies indicate that SOX17 acts as a key factor in activating the arterial program and HOXA expression in HE.

### SOX17 Promotes Arterial Program in HE Through Activation of NOTCH Signaling

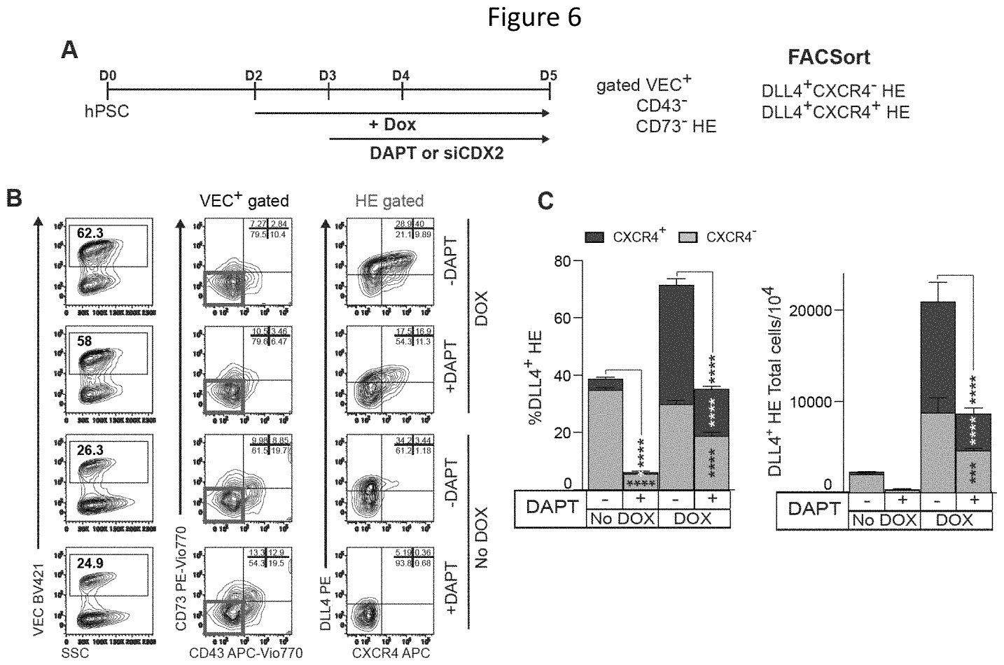

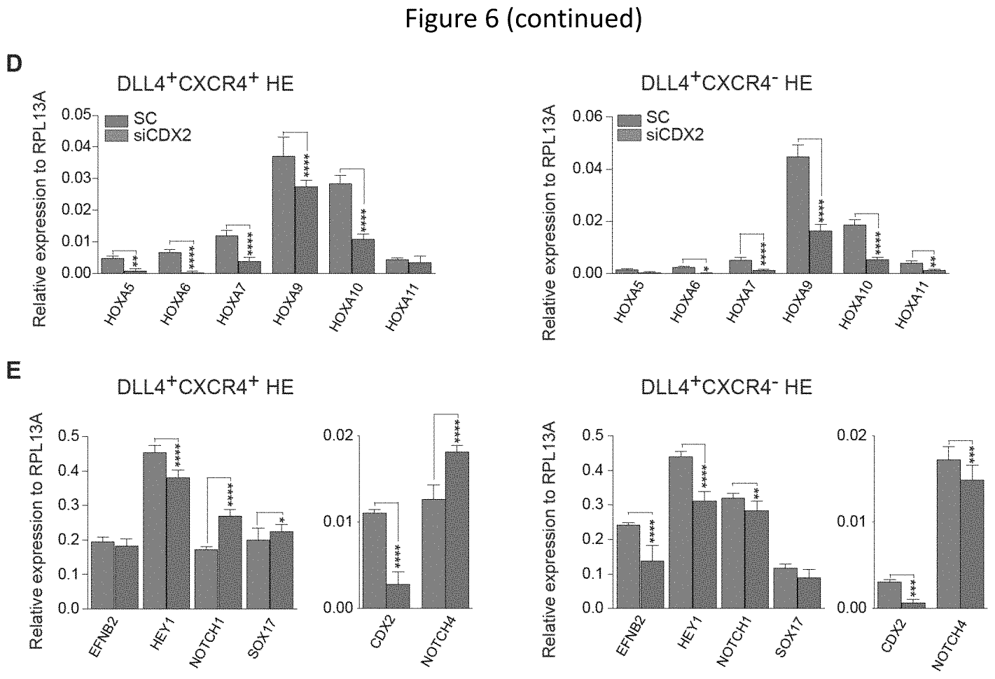

To determine whether SOX17 induction promotes arterial specification through activation of NOTCH signaling, we evaluated hematopoiesis following SOX17 upregulation in the presence of the NOTCH signaling inhibitor N—[N-(3,5-difluorophenacetyl)-L-alanyl]-S-phenylglycine t-butyl ester (DAPT) (FIG. 6A). As shown in FIG. 6B-C, treatment of hESC cultures with DAPT almost completely abrogated formation of AHE in DOX− conditions and markedly reduced the effect of SOX17 on AHE formation in DOX+ conditions confirming the important role of NOTCH activation in SOX17-mediated promotion of the arterial hemogenic program.

### SOX17 Mediates HOXA Gene Expression Through Activation of CDX2 Expression in HE

Despite significant upregulation of HOXA cluster genes in SOX17-expressing HE, our molecular profiling studies lacked of evidence for their direct regulation by SOX17. However, we found that SOX17 binds to and increases ATAC-seq counts of H3K27ac levels at the CDX2 promoter, along with the upregulation of CDX2 gene expression (FIG. 4H). The CDX2 gene is known to play a critical role in regulating HOX genes, including HOXA genes (Charite et al., 1998; Subramanian et al., 1995; van den Akker et al., 2002), particularly the HOXA genes necessary for the specification of hematopoietic cells during vertebrate embryogenesis (Davidson et al., 2003; Davidson and Zon, 2006). To find out whether the SOX17 effect on HOXA expression is mediated by CDX2, we differentiated the iSOX17-hESC line with DOX treatment from D2-5 and treated the cultures with CDX2 siRNA from D3-5. On D5, CXCR4⁺ and CXCR4⁻ AHE subsets were analyzed for HOXA cluster and arterial gene expression (FIG. 6A). We found that inhibition of CDX2 with siRNA decreased expression of HOXA genes in both AHE subsets, but had variable effects on the expression of arterial markers (FIG. 6D-E). In the DLL4⁺CXCR4⁺ population, CDX2 siRNA increased NOTCH1 and NOTCH4 expression, while in the DLL4⁺CXCR4⁻ subset, their expression decreased. The effect of siCDX2 on SOX17 expression was minimal (FIG. 6E).

Overall, these observations indicate that the effect of SOX17 expression on establishing HOXA signature in HE is mediated through CDX2 signaling.

### Discussion:

The critical role of SOX17 in HSC development has been well-recognized (Clarke et al., 2013; Kim et al., 2007; Lizama et al., 2015; Nobuhisa et al., 2014; Saito et al., 2018). Although SOX17 regulates multiple steps along the HSC developmental path, including HE specification, EHT, and HSC maintenance and expansion, the stage-specific molecular mechanisms of SOX17 are not well understood. Previously, overexpression of Sox17 was demonstrated to decrease cell numbers within the IAHC, while loss of Sox17 had the opposite effect (Lizama et al., 2015). However, the HSC potential of IAHC of manipulated AGM cells has not been characterized. The majority of cells within IAHC are differentiated hematopoietic cells, with only two of those cells possessing HSC potential (Kumaravelu et al., 2002; Solaimani Kartalaei et al., 2015). Thus, increase in IAHC cells following Sox17 downregulation could be associated with increase of lineage-committed progenitors which is accompanied by impaired HSC generation. This hypothesis is supported by observation that Sox17 loss leads to loss of CD45⁺VEC⁺ HSCs in AGM and fetal liver (Clarke et al., 2013; Kim et al., 2007). Demonstration of reduced multilineage CFC potential following knockdown of Sox17 in CD45^{low}CD117^{high} cells from IAHC and expansion of undifferentiated hematopoietic cells following Sox17 overexpression (Nobuhisa et al., 2014) supports this hypothesis. In addition to regulating EHT and HSCs, SOX17 is essential for arterial specification and HE formation in the AGM (Clarke et al., 2013; Corada et al., 2013). Murine studies revealed that Sox17 effects are mediated through NOTCH signaling (Clarke et al., 2013; Lizama et al., 2015), while no effect of SOX17 on NOTCH signaling was observed in hESC cultures (Nakajima-Takagi et al., 2013).

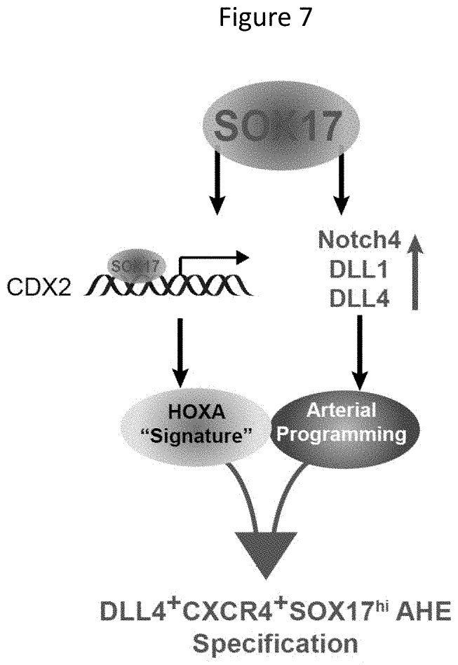

A prior study revealed that Sox17 overexpression in hESC-derived during EHT expands VEC⁺CD34⁺CD43⁺CD45^{-/low} cells with hematopoietic colony-forming potential (Nakajima-Takagi et al., 2013). In our study, we focused on defining the cellular and molecular pathways by which SOX17 regulates the earliest stages of HE specification and diversification from the mesoderm. Previously, we and others demonstrated that emerging HE cells lack arterial or venous characteristics (Ditadi et al., 2015; Uenishi et al., 2018) and express high levels of the mesodermal gene HAND1 (Uenishi et al., 2018). Therefore, we defined these cells as immature/primordial HE (Uenishi et al., 2018). When primordial HE cells get exposed to NOTCH signaling, they undergo arterial specification and formation of DLL4⁺CXCR4^{+/-} AHE (Park et al., 2018b; Uenishi et al., 2018). Here, we show that SOX17 plays a critical role in specifying of AHE by upregulation of NOTCH4, DLL1 and DLL4 eventually leading to the formation of AHE with the DLL4⁺CXCR4⁺ phenotype typical of AHE at sites of HSC emergence, but not yolk sac AHE, which expresses DLL4, but not CXCR4 (McGrath et al., 1999; Venkatesh et al., 2008; Werner et al., 2020). Along with activating the arterial program, SOX17 is essential for expressing HOXA genes in AHE (FIG. 7). This integrated effect on arterial programming and HOXA expression is unique for SOX17 since it was not observed following overexpression of ETS1, an arterial program specific gene from our prior studies (Park et al., 2018b).

We found that SOX17 binds directly to the CDX2 promoter and increases chromatin accessibility and levels of H3K27ac activating histone modification leading to upregulated CDX2 expression. CDX2 knockdown with siRNA revokes SOX17-mediated effects on HOXA genes, thus demonstrating the critical role of a SOX17-CDX2 axis in establishing HOXA pattern in AHE. Genes of the CDX family (CDX1, CDX2 and CDX4) are well-known master regulators of HOX genes that mediate anterior-posterior patterning (Charite et al., 1998; Subramanian et al., 1995; van den Akker et al., 2002). In Wnt-activated epiblast stem cells, CDX2 binds to all four HOX cluster genes, including HOXA genes and is required for opening up the HOX cis-sequences (Neijts et al., 2017). Deficiency of cdx1 and cdx4 results in severe blood defects and altered expression of HOX genes in zebrafish (Davidson et al., 2003; Davidson and Zon, 2006). Similarly, impaired hematopoiesis from Cdx1, Cdx2 or Cdx4-deficient ESCs was observed in murine studies (Wang et al., 2008). Although ectopic expression of CDX4 enhanced definitive hematopoiesis from human and murine ESCs (Creamer et al., 2017; Wang et al., 2005), and hematopoietic engraftment in adult mice from murine ESCs (Wang et al., 2005), Cdx1 and Cdx4 double mutant mice were viable and did not show any hematopoietic defect (van Nes et al., 2006), which could be due to the observed functional redundancy of genes within the Cdx family (Davidson and Zon, 2006; Wang et al., 2008). It has been demonstrated that Cdx2 is the predominant Cdx gene expressed the AGM HE, while the expression of other Cdx genes is substantially lower (Gao et al., 2018). Cdx2 deficiency caused the most significant impairment in blood production from mouse ESCs (Wang et al., 2008) and Cdx1-Cdx2 compound conditional null mice failed to produce any blood at E11.5 (Foley et al., 2019), suggesting that among the Cdx family, Cdx2 is the most critical factor required for establishing hematopoiesis. Our finding of direct regulation of CDX2 expression by SOX17 provides an insight into the mechanisms responsible for establishing a CDX-HOXA pathway required for the formation of definitive AGM-like HE and lympho-myeloid hematopoiesis from hPSCs. Although previous studies with mouse ESCs found that overexpression of Cdx2 inhibits hematopoietic differentiation (McKinney-Freeman et al., 2008), such effect was not observed following upregulation of CDX2 by SOX17 in hESCs. This could be explained by differences in levels of upregulation or molecular programs activated by upregulation of CDX2 alone or in the context of SOX17 overexpression.

The de novo production of hematopoietic stem cells (HSCs) with robust multilineage reconstitution potential from human pluripotent stem cells (hPSCs) has long been sought after, but remains an elusive goal. Recent advances in understanding the molecular differences between HE and HSCs developed in vivo and their phenotypic counterparts produced from PSCs in vitro, have revealed that deficiencies in NOTCH and HOXA signaling are major factors responsible for aberrant functionality of PSC-derived hemogenic progenitors (Dou et al., 2016; Doulatov et al., 2013; McKinney-Freeman et al., 2012; Ng et al., 2016; Salvagiotto et al., 2008; Sugimura et al., 2017). It is well-established that NOTCH signaling is essential for arterial specification and development of HSCs (Burns et al., 2005; Kumano et al., 2003). Knock-out of the HOXA cluster in adult mice severely compromised HSC activity (Lebert-Ghali et al., 2016). In humans, HOXA5 and HOXA7 were shown to be critical for the expansion of engraftable fetal liver HSCs (Dou et al., 2016). Although overexpression of single or multiple medial HOXA genes in PSC-derived CD34⁺ cells was insufficient to confer HSC function (Dou et al., 2016; Ramos-Mejia et al., 2014), overexpression of HOXA5, HOXA9, and HOXA10 along with ERG, LCOR, RUNX1 and SPI1 hPSC-derived HE was capable of generating engraftable hematopoietic cells (Sugimura et al., 2017). In the present study, we provided a compelling evidence that SOX17 is a master regulator that integrates HOXA and arterial signature in HE through modulation of CDX2 signaling. This important finding may contribute to the strategic targeting of NOTCH and HOXA pathways to enhance lymphoid and engraftable hematopoietic cell production from hPSCs for the therapies of hematologic and oncologic diseases, including off-the-shelf immunotherapies.

### Example 2

### Expansion of Hematopoietic Progenitors with Myeloid and T Lymphoid Potentials

SOX17 expands lymphomyeloid progenitors generated from day 4 hemogenic endothelium.

To evaluate the role of SOX17 in expansion of hematopoietic progenitors, we isolated day 4 HE from DOX+ and DOX− cultures (phase I DOX treatment) and cultured these cells on OP9 or OP9-DLL4. After 5 days, floating hematopoietic progenitors were collected and cultured with or without DOX for additional 5 days (phase II DOX treatment) and evaluated for CFC and T lymphoid potential (FIG. 14). We found that hematopoietic progenitors expanded without DOX lost hematopoietic CFC and T cell potential completely. In contrast hematopoietic progenitors treated with DOX in suspension cultures (day 4+5 through day 4+5+5) retained CFC and T cell potential. We also noted a significant increase myeloid CFCs and T cell potential following phase II DOX treatment when HE were isolated from phase I DOX-treated cultures. This effect was especially pronounced in HE cultured on OP9-DLL4 (FIGS. 14B and C). As compare to phase I only treated cells, T cell production from hematopoietic progenitors produced from phase I and phase II treated cells was increased by 11-16 fold.

### REFERENCES

Bailey, T. L. and Machanick, P. (2012). Inferring direct DNA binding from ChIP-seq. Nucleic Acids Res 40 e128
Buenrostro, J. D., Giresi, P. G., Zaba, L. C., Chang, H. Y., and Greenleaf, W. J. (2013). Transposition of native chromatin for fast and sensitive epigenomic profiling of open chromatin, DNA-binding proteins and nucleosome position. Nat Methods 10, 1213-1218.
Burns, C. E., Traver, D., Mayhall, E., Shepard, J. L., and Zon, L. I. (2005). Hematopoietic stem cell fate is established by the Notch-Runx pathway. Genes Dev 19, 2331-2342.
Charite, J., de Graaff, W., Consten, D., Reijnen, M. J., Korving, J., and Deschamps, J. (1998). Transducing positional information to the Hox genes: critical interaction of cdx gene products with position-sensitive regulatory elements. Development 125, 4349-4358.
Choi, K. D., Vodyanik, M. A., Togarrati, P. P., Suknuntha, K., Kumar, A., Samarjeet, F., Probasco, M. D., Tian, S., Stewart, R., Thomson, J. A., et al. (2012). Identification of the hemogenic endothelial progenitor and its direct precursor in human pluripotent stem cell differentiation cultures. Cell Rep 2, 553-567.
Chong, D. C., Koo, Y., Xu, K., Fu, S., and Cleaver, 0. (2011). Stepwise arteriovenous fate acquisition during mammalian vasculogenesis. Dev Dyn 240, 2153-2165.
Clarke, R. L., Yzaguirre, A. D., Yashiro-Ohtani, Y., Bondue, A., Blanpain, C., Pear, W. S., Speck, N. A., and Keller, G. (2013). The expression of Sox17 identifies and regulates haemogenic endothelium. Nat Cell Biol 15, 502-510.
Corada, M., Orsenigo, F., Morini, M. F., Pitulescu, M. E., Bhat, G., Nyqvist, D., Breviario, F., Conti, V., Briot, A., Iruela-Arispe, M. L., et al. (2013). Sox17 is indispensable for acquisition and maintenance of arterial identity. Nat Commun 4, 2609.
Creamer, J. P., Dege, C., Ren, Q., Ho, J. T. K., Valentine, M. C., Druley, T. E., and Sturgeon, C. M. (2017). Human definitive hematopoietic specification from pluripotent stem cells is regulated by mesodermal expression of CDX4. Blood 129, 2988-2992.
Davidson, A. J., Ernst, P., Wang, Y., Dekens, M. P., Kingsley, P. D., Palis, J., Korsmeyer, S. J., Daley, G. Q., and Zon, L. I. (2003). cdx4 mutants fail to specify blood progenitors and can be rescued by multiple hox genes. Nature 425, 300-306.
Davidson, A. J., and Zon, L. I. (2006). The caudal-related homeobox genes cdx1a and cdx4 act redundantly to regulate hox gene expression and the formation of putative hematopoietic stem cells during zebrafish embryogenesis. Dev Biol 292, 506-518.
Ditadi, A., Sturgeon, C. M., Tober, J., Awong, G., Kennedy, M., Yzaguirre, A. D., Azzola, L., Ng, E. S., Stanley, E. G., French, D. L., et al. (2015). Human definitive haemogenic endothelium and arterial vascular endothelium represent distinct lineages. Nat Cell Biol 17, 580-591.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinfromatics 29, 15-21.
Dou, D. R., Calvanese, V., Sierra, M. I., Nguyen, A. T., Minasian, A., Saarikoski, P., Sasidharan, R., Ramirez, C. M., Zack, J. A., Crooks, G. M., et al. (2016). Medial HOXA genes demarcate haematopoietic stem cell fate during human development. Nat Cell Biol 18, 595-606.
Doulatov, S., Vo, L. T., Chou, S. S., Kim, P. G., Arora, N., Li, H., Hadland, B. K., Bernstein, I. D., Collins, J. J., Zon, L. I., et al. (2013). Induction of multipotential hematopoietic progenitors from human pluripotent stem cells via respecification of lineage-restricted precursors. Cell Stem Cell 13, 459-470.
Duarte, A., Hirashima, M., Benedito, R., Trindade, A., Diniz, P., Bekman, E., Costa, L., Henrique, D., and Rossant, J. (2004). Dosage-sensitive requirement for mouse Dll4 in artery development. Genes Dev 18, 2474-2478.
Dzierzak, E., and Medvinsky, A. (1995). Mouse embryonic hematopoiesis. Trends Genet 11, 359-366.
Foley, T. E., Hess, B., Savory, J. G. A., Ringuette, R., and Lohnes, D. (2019). Role of Cdx factors in early mesodermal fate decisions. Development 146.
Gao, L., Tober, J., Gao, P., Chen, C., Tan, K., and Speck, N. A. (2018). RUNX1 and the endothelial origin of blood. Exp Hematol 68, 2-9.
Gordon-Keylock, S., Sobiesiak, M., Rybtsov, S., Moore, K., and Medvinsky, A. (2013). Mouse extraembryonic arterial vessels harbor precursors capable of maturing into definitive HSCs. Blood 122, 2338-2345.
Hadland, B. K., Huppert, S. S., Kanungo, J., Xue, Y., Jiang, R., Gridley, T., Conlon, R. A., Cheng, A. M., Kopan, R., and Longmore, G. D. (2004). A requirement for Notch1 distinguishes 2 phases of definitive hematopoiesis during development. Blood 104, 3097-3105.
Herman, A. M., Rhyner, A. M., Devine, W. P., Marrelli, S. P., Bruneau, B. G., and Wythe, J. D. (2018). A novel reporter allele for monitoring Dll4 expression within the embryonic and adult mouse. Biol Open 7.
Hou, Z., Jiang, P., Swanson, S. A., Elwell, A. L., Nguyen, B. K., Bolin, J. M., Stewart, R., and Thomson, J. A. (2015). A cost-effective RNA sequencing protocol for large-scale gene expression studies. Sci Rep 5, 9570.
Hu, Y., and Smyth, G. K. (2009). ELDA: extreme limiting dilution analysis for comparing depleted and enriched populations in stem cell and other assays. J Immunol Methods 347, 70-78.
Kennedy, M., Awong, G., Sturgeon, C. M., Ditadi, A., LaMotte-Mohs, R., Zuniga-Pflucker, J. C., and Keller, G. (2012). T lymphocyte potential marks the emergence of definitive hematopoietic progenitors in human pluripotent stem cell differentiation cultures. Cell Rep 2, 1722-1735.
Kim, I., Saunders, T. L., and Morrison, S. J. (2007). Sox17 dependence distinguishes the transcriptional regulation of fetal from adult hematopoietic stem cells. Cell 130, 470-483.
Kumano, K., Chiba, S., Kunisato, A., Sata, M., Saito, T., Nakagami-Yamaguchi, E., Yamaguchi, T., Masuda, S., Shimizu, K., Takahashi, T., et al. (2003). Notch1 but not Notch2 is essential for generating hematopoietic stem cells from endothelial cells. Immunity 18, 699-711.
Kumaravelu, P., Hook, L., Morrison, A. M., Ure, J., Zhao, S., Zuyev, S., Ansell, J., and Medvinsky, A. (2002). Quantitative developmental anatomy of definitive haematopoietic stem cells/long-term repopulating units (HSC/RUs): role of the aorta-gonad-mesonephros (AGM) region and the yolk sac in colonisation of the mouse embryonic liver. Development 129, 4891-4899.
Lebert-Ghali, C. E., Fournier, M., Kettyle, L., Thompson, A., Sauvageau, G., and Bijl, J. J. (2016). Hoxa cluster genes determine the proliferative activity of adult mouse hematopoietic stem and progenitor cells. Blood 127, 87-90.
Li, B. and Dewey, C. N. (2011). RSEM: accurate transcript quantification from RNA-Seq data with or without a reference genome. BMC Bioinformatics 12, 323
Li, H. and Durbin, R. (2009). Fast and accurate short read alignment with Burrows-Wheeler transform. Bioinformatics 25, 1754-1960
Liao, W. P., Uetzmann, L., Burtscher, I., and Lickert, H. (2009). Generation of a mouse line expressing Sox17-driven Cre recombinase with specific activity in arteries. Genesis 47, 476-483.
Liu, M., Zhang, L., Marsboom, G., Jambusaria, A., Xiong, S., Toth, P. T., Benevolenskaya, E. V., Rehman, J., and Malik, A. B. (2019). Sox17 is required for endothelial regeneration following inflammation-induced vascular injury. Nat Commun 10, 2126.
Lizama, C. O., Hawkins, J. S., Schmitt, C. E., Bos, F. L., Zape, J. P., Cautivo, K. M., Borges Pinto, H., Rhyner, A. M., Yu, H., Donohoe, M. E., et al. (2015). Repression of arterial genes in hemogenic endothelium is sufficient for haematopoietic fate acquisition. Nat Commun 6, 7739.
McGrath, K. E., Koniski, A. D., Maltby, K. M., McGann, J. K., and Palis, J. (1999). Embryonic expression and function of the chemokine SDF-1 and its receptor, CXCR4. Dev Biol 213, 442-456.
McKinney-Freeman, S., Cahan, P., Li, H., Lacadie, S. A., Huang, H. T., Curran, M., Loewer, S., Naveiras, O., Kathrein, K. L., Konantz, M., et al. (2012). The transcriptional landscape of hematopoietic stem cell ontogeny. Cell Stem Cell 11, 701-714.
McKinney-Freeman, S. L., Lengerke, C., Jang, I. H., Schmitt, S., Wang, Y., Philitas, M., Shea, J., and Daley, G. Q. (2008). Modulation of murine embryonic stem cell-derived CD41+c-kit+ hematopoietic progenitors by ectopic expression of Cdx genes. Blood 111, 4944-4953.
Nakajima-Takagi, Y., Osawa, M., Oshima, M., Takagi, H., Miyagi, S., Endoh, M., Endo, T. A., Takayama, N., Eto, K., Toyoda, T., et al. (2013). Role of SOX17 in hematopoietic development from human embryonic stem cells. Blood 121, 447-458.
Neijts, R., Amin, S., van Rooijen, C., and Deschamps, J. (2017). Cdx is crucial for the timing mechanism driving colinear Hox activation and defines a trunk segment in the Hox cluster topology. Dev Biol 422, 146-154.
Ng, E. S., Azzola, L., Bruveris, F. F., Calvanese, V., Phipson, B., Vlahos, K., Hirst, C., Jokubaitis, V. J., Yu, Q. C., Maksimovic, J., et al. (2016). Differentiation of human embryonic stem cells to HOXA(+) hemogenic vasculature that resembles the aorta-gonad-mesonephros. Nat Biotechnol 34, 1168-1179.
Nobuhisa, I., Osawa, M., Uemura, M., Kishikawa, Y., Anani, M., Harada, K., Takagi, H., Saito, K., Kanai-Azuma, M., Kanai, Y., et al. (2014). Sox17-mediated maintenance of fetal intra-aortic hematopoietic cell clusters. Mol Cell Biol 34, 1976-1990.
Park, M. A., Jung, H. S., and Slukvin, I. (2018a). Genetic Engineering of Human Pluripotent Stem Cells Using PiggyBac Transposon System. Curr Protoc Stem Cell Biol 47, e63.

Park, M. A., Kumar, A., Jung, H. S., Uenishi, G., Moskvin, O. V., Thomson, J. A., and Slukvin, II (2018b). Activation of the Arterial Program Drives Development of Definitive Hemogenic Endothelium with Lymphoid Potential. Cell Rep 23, 2467-2481.
Ramos-Mejia, V., Navarro-Montero, O., Ayllon, V., Bueno, C., Romero, T., Real, P. J., and Menendez, P. (2014). HOXA9 promotes hematopoietic commitment of human embryonic stem cells. Blood 124, 3065-3075.
Robert-Moreno, A., Espinosa, L., de la Pompa, J. L., and Bigas, A. (2005). RBPjkappa-dependent Notch function regulates Gata2 and is essential for the formation of intra-embryonic hematopoietic cells. Development 132, 1117-1126.
Saito, K., Nobuhisa, I., Harada, K., Takahashi, S., Anani, M., Lickert, H., Kanai-Azuma, M., Kanai, Y., and Taga, T. (2018). Maintenance of hematopoietic stem and progenitor cells in fetal intra-aortic hematopoietic clusters by the Sox17-Notch1-Hes1 axis. Exp Cell Res 365, 145-155.
Salvagiotto, G., Zhao, Y., Vodyanik, M., Ruotti, V., Stewart, R., Marra, M., Thomson, J., Eaves, C., and Slukvin, I. (2008). Molecular profiling reveals similarities and differences between primitive subsets of hematopoietic cells generated in vitro from human embryonic stem cells and in vivo during embryogenesis. Exp Hematol 36, 1377-1389.
Serrano, A. G., Gandillet, A., Pearson, S., Lacaud, G., and Kouskoff, V. (2010). Contrasting effects of Sox17- and Sox18-sustained expression at the onset of blood specification. Blood 115, 3895-3898.

Solaimani Kartalaei, P., Yamada-Inagawa, T., Vink, C. S., de Pater, E., van der Linden, R., Marks-Bluth, J., van der Sloot, A., van den Hout, M., Yokomizo, T., van Schaick-Solerno, M. L., et al. (2015). Whole-transcriptome analysis of endothelial to hematopoietic stem cell transition reveals a requirement for Gpr56 in HSC generation. J Exp Med 212, 93-106.
Subramanian, V., Meyer, B. I., and Gruss, P. (1995). Disruption of the murine homeobox gene Cdx1 affects axial skeletal identities by altering the mesodermal expression domains of Hox genes. Cell 83, 641-653.
Sugimura, R., Jha, D. K., Han, A., Soria-Valles, C., da Rocha, E. L., Lu, Y. F., Goettel, J. A., Serrao, E., Rowe, R. G., Malleshaiah, M., et al. (2017). Haematopoietic stem and progenitor cells from human pluripotent stem cells. Nature 545, 432-438.
Uenishi, G., Theisen, D., Lee, J. H., Kumar, A., Raymond, M., Vodyanik, M., Swanson, S., Stewart, R., Thomson, J., and Slukvin, I. (2014). Tenascin C promotes hematoendothelial development and T lymphoid commitment from human pluripotent stem cells in chemically defined conditions. Stem cell reports 3, 1073-1084.
Uenishi, G. I., Jung, H. S., Kumar, A., Park, M. A., Hadland, B. K., McLeod, E., Raymond, M., Moskvin, O., Zimmerman, C. E., Theisen, D. J., et al. (2018). NOTCH signaling specifies arterial-type definitive hemogenic endothelium from human pluripotent stem cells. Nat Commun 9, 1828.
van den Akker, E., Forlani, S., Chawengsaksophak, K., de Graaff, W., Beck, F., Meyer, B. I., and Deschamps, J. (2002). Cdx1 and Cdx2 have overlapping functions in anteroposterior patterning and posterior axis elongation. Development 129, 2181-2193.
van Nes, J., de Graaff, W., Lebrin, F., Gerhard, M., Beck, F., and Deschamps, J. (2006). The Cdx4 mutation affects axial development and reveals an essential role of Cdx genes in the ontogenesis of the placental labyrinth in mice. Development 133, 419-428.
Venkatesh, D. A., Park, K. S., Harrington, A., Miceli-Libby, L., Yoon, J. K., and Liaw, L. (2008). Cardiovascular and hematopoietic defects associated with Notch1 activation in embryonic Tie2-expressing populations. Circ Res 103, 423-431.
Vodyanik, M. A., Yu, J., Zhang, X., Tian, S., Stewart, R., Thomson, J. A., and Slukvin, II (2010). A mesoderm-derived precursor for mesenchymal stem and endothelial cells. Cell Stem Cell 7, 718-729.
Wang, Y., Yabuuchi, A., McKinney-Freeman, S., Ducharme, D. M., Ray, M. K., Chawengsaksophak, K., Archer, T. K., and Daley, G. Q. (2008). Cdx gene deficiency compromises embryonic hematopoiesis in the mouse. Proc Natl Acad Sci USA 105, 7756-7761.
Wang, Y., Yates, F., Naveiras, O., Ernst, P., and Daley, G. Q. (2005). Embryonic stem cell-derived hematopoietic stem cells. Proc Natl Acad Sci USA 102, 19081-19086.
Werner, Y., Mass, E., Ashok Kumar, P., Ulas, T., Handler, K., Horne, A., Klee, K., Lupp, A., Schutz, D., Saaber, F., et al. (2020). Cxcr4 distinguishes HSC-derived monocytes from microglia and reveals monocyte immune responses to experimental stroke. Nat Neurosci 23, 351-362.
Yamamizu, K., Matsunaga, T., Uosaki, H., Fukushima, H., Katayama, S., Hiraoka-Kanie, M., Mitani, K., and Yamashita, J. K. (2010). Convergence of Notch and beta-catenin signaling induces arterial fate in vascular progenitors. J Cell Biol 189, 325-338.
Zhang, Y., Liu, T., Meyer, C. A., Eeckhoute, J., Johnson, D. S., Bernstein, B. E., Nusbaum, C., Myers, R. M., Brown, M., Li, W., and Liu, X. S. (2008). Model-based analysis of ChIP-Seq (MACS). Genome Biol 9, R137

What is claimed:

1. A method of enhancing HOXA gene expression and arterial specification of hemogenic endothelium in differentiating human pluripotent stem cells (hPSCs), the method comprising:
(a) introducing an inducible SOX17 transgene into a population of hPSCs;
(b) culturing the hPSCs for at least two days under conditions to differentiate the hPSC into KDR⁺ mesoderm cells; and
(c) inducing expression of the SOX17 transgene in the KDR⁺ mesoderm cells and culturing for at least two days, such that DLL4⁺CXCR4⁺ arterial hemogenic endothelium (AHE) cells are obtained.

2. The method of claim 1, wherein the population of hPSCs expresses the SOX17 transgene for two to four days.

3. The method of claim 1, wherein step (a) comprises transducing the hPSCs with a vector comprising the SOX17 transgene.

4. The method of claim 3, wherein the vector comprises an inducible promoter operably linked to the SOX17 transgene.

5. The method of claim 4, wherein the vector comprises SEQ ID NO:1.

6. The method of claim 1, wherein the SOX17 transgene comprises SEQ ID NO:58.

7. The method of claim 1, wherein the AHE cells express one or more arterial markers selected from the group consisting of EFNB2, DLL4, NOTCH4, CXCR4, and HEY1.

8. The method of claim 1, wherein the AHE cells express one or more HOXA genes selected from the group consisting of HOXA5, HOXA7, HOXA9, HOXA10, and HOX11.

9. The method of claim 1, wherein the AHE cells express CDX2.

10. The method of claim 1, wherein the hPSCs are embryonic stem cells (ESCs) or induced pluripotent stem cells (iPSCs).

11. The method claim 1, wherein the cells are further differentiated into a myeloid cell line or lymphoid cell line.

12. The method of claim 11, wherein the method further comprises:
(d) culturing the hematopoietic progenitors produced in SOX17 overexpression cultures in the presence of cells overexpressing DLL4 in media comprising SCF, FLT3L and IL-7 for at least two weeks to produce CD4+CD8+ T cells.

13. The method of claim 11, wherein the method further comprises:
(d) inducing expression of the SOX17 transgene in the mesodermal cells of step (c) such that DLL4⁺CXCR4⁺ arterial hemogenic endothelium (AHE) cells are obtained;
(e) culturing AHE in the presence of cells overexpressing DLL4 for a sufficient time to differentiate to floating hematopoietic progenitors, and
(f) collecting the floating hematopoietic progenitor cells and culturing in the presence of DLL4 in media comprising SCF, FLT3L and IL-7 for at least two weeks to produce CD4+CD8+ T cells.

14. A cell population produced by the method of claim 1.

15. The cell population of claim 14, wherein the cell population is at least 90% DLL4⁺CXCR4⁺ AHE cells.

16. An hPSC population that comprises a SOX17 transgene and is capable of differentiating into DLL4⁺CXCR4⁺ AHE cells.

17. The hPSC population of claim 16, wherein the cells comprise a vector comprising the SOX17 transgene.

18. The hPSC population of claim 17, wherein the vector comprises an inducible promoter operably linked to the SOX17 transgene.

19. The hPSC population of claim 18, wherein the vector comprises SEQ ID NO:1.

20. The hPSC population of any one of claim 16, wherein the SOX17 transgene comprises SEQ ID NO:58.

21. An isolated in vitro population of DLL4⁺CXCR4⁺ AHE cells differentiated from an hPSC population comprising a SOX17 transgene.

22. An isolated in vitro T cell population differentiated from the AHE cells of claim 21.

23. The isolated in vitro T cell population of claim 22, wherein the T cell population comprises more than 90% CD4⁺CD8⁺ T cells.

24. The isolated in vitro T cell population of claim 22, wherein the T cells are engineered to express an exogenous chimeric antigen receptor (CAR).

25. A method of expansion of hematopoietic progenitors comprising
a) generating hemogenic endothelium (HE) cells in presence of SOX17 upregulation
b) culturing the HE cells on OP9 or OP9-DLL4 cells in medium comprising FLT3L, TPO SCF, IL6, and IL3 for at least an additional 5 days;
c) collecting the floating hematopoietic progenitor cells (HP); and
d) culturing the HPs of step c in medium comprising FLT3L, TPO SCF, IL6, and IL3 for at least an additional 5 days to expand HPs with myeloid and lymphoid potential.

26. The method of claim 25, further comprising:
(e) passaging the cells of step (d) for at least two weeks in medium comprising SCF, FLT3L and IL-7 for at least two weeks to produce CD4+CD8+ T cells.


SEQUENCE LISTING (58 sequences)

SEQ ID NO: 1 - DNA, 4441 bp, Artificial Sequence
  Synthetic - SOX17 vector
  misc_feature (330)..(1571): human SOX17
cgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggaggagctagcatgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtggtatacggaagcggagctactaacttcagcctgctgaagcaggctggagacgtggaggagaaccctggacccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaatcatgggctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgatgcatgcgcgaggcagccggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttagattcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgctagttgctctagatggccgcgaccacgtgttcacgacacctgaaatggaagaaaaaaactttgaaccactgtctgaggcttgagaatgaaccaagatccaaactcaaaaagggcaaattccaacgagaattacatcaagtgccaagctggcctaacttcagtctccacccactcagtgtggggaaactccatcgcataaaacccctccccccaacctaaagacgacgtactccaaaagctccagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagccccctggagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccggccgcgggcccaaaaaccgaaataccagtgtgcagatcctggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctcctaaagcgcaaggcccgcccctggctccagctcgggatcaagaatcacgtactggagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgaagctag

SEQ ID NO: 2 - DNA, 20 bp, Artificial Sequence
  Synthetic - Single guide RNA 1 (sgRNA1) for generation of SOX17-/- knockout H9 ESC line
gttcatcggccgccggatac

SEQ ID NO: 3 - DNA, 20 bp, Artificial Sequence
  Synthetic - Single guide RNA 2 (sgRNA2) for generation of SOX17-/- knockout H9 ESC line
ttcacctgcttgcgccgccg

SEQ ID NO: 4 - DNA, 20 bp, Artificial Sequence
  Synthetic - Primer
tctgtccctctctgctttct

SEQ ID NO: 5 - DNA, 21 bp, Artificial Sequence
  Synthetic - Primer
ctgcctggcctacatgttatc

SEQ ID NO: 6 - DNA, 22 bp, Artificial Sequence
  Synthetic - Primer
ccgaacagggacttgtttagag

SEQ ID NO: 7 - DNA, 22 bp, Artificial Sequence
  Synthetic - Primer
aggttggctctggcatttatag

SEQ ID NO: 8 - DNA, 20 bp, Artificial Sequence
  Synthetic - Primer
tcagtggctgacctcctctt

SEQ ID NO: 9 - DNA, 20 bp, Artificial Sequence
  Synthetic - Primer
cttggcctttgactgttggt

SEQ ID NO: 10 - DNA, 20 bp, Artificial Sequence
  Synthetic - Primer
cagtgggcagcgaagctaca

SEQ ID NO: 11 - DNA, 22 bp, Artificial Sequence
  Synthetic - Primer
acaggcagtggtagccatcctc

SEQ ID NO: 12 - DNA, 21 bp, Artificial Sequence
  Synthetic - Primer
ctcctcaactgtgccaaacca

SEQ ID NO: 13 - DNA, 20 bp, Artificial Sequence
  Synthetic - Primer
ggttatccaggccctccaaa

SEQ ID NO: 14 - DNA, 22 bp, Artificial Sequence
  Synthetic - Primer
ggactatcggagtttgggattt

SEQ ID NO: 15 - DNA, 20 bp, Artificial Sequence
  Synthetic - Primer
tgggaagcgtagttgttgag

SEQ ID NO: 16 - DNA, 23 bp, Artificial Sequence
  Synthetic - Primer
ttcaaggcagctcggtaactgac

SEQ ID NO: 17 - DNA, 23 bp, Artificial Sequence
  Synthetic - Primer
catactgatgcactgctggatgg

SEQ ID NO: 18 - DNA, 21 bp, Artificial Sequence
  Synthetic - Primer
ccaacagaaacatgccagaag

SEQ ID NO: 19 - DNA, 22 bp, Artificial Sequence
  Synthetic - Primer
ctcgcctttcgctatatcctac

SEQ ID NO: 20 - DNA, 21 bp, Artificial Sequence
  Synthetic - Primer
gatgcagtttcacccagtttg

SEQ ID NO: 21 - DNA, 22 bp, Artificial Sequence
  Synthetic - Primer
gattgtggtgagtgtgtctgta

SEQ ID NO: 22 - DNA, 22 bp, Artificial Sequence
  Synthetic - Primer
gatggagccatgggaagattac

SEQ ID NO: 23 - DNA, 24 bp, Artificial Sequence
  Synthetic - Primer
catagggaggaggctgagatatag

SEQ ID NO: 24 - DNA, 22 bp, Artificial Sequence
  Synthetic - Primer
ggagaaggagttccacttcaat

SEQ ID NO: 25 - DNA, 22 bp, Artificial Sequence
  Synthetic - Primer
ggtctttcttccacttcatcct

SEQ ID NO: 26 - DNA, 22 bp, Artificial Sequence
  Synthetic - Primer
gttcctgtctcaatagctccaa

SEQ ID NO: 27 - DNA, 23 bp, Artificial Sequence
  Synthetic - Primer
gtgtctcatcaagtcacctctac

SEQ ID NO: 28 - DNA, 20 bp, Artificial Sequence
  Synthetic - Primer
aaagcactccatgacgaagg

SEQ ID NO: 29 - DNA, 20 bp, Artificial Sequence
  Synthetic - Primer
gtctggtagcgcgtgtaggt

SEQ ID NO: 30 - DNA, 20 bp, Artificial Sequence
  Synthetic - Primer
aggtccaggatcagggtatt

SEQ ID NO: 31 - DNA, 22 bp, Artificial Sequence
  Synthetic - Primer
ccagagaaggagggattgattc

SEQ ID NO: 32 - DNA, 20 bp, Artificial Sequence
  Synthetic - Primer
gcgccttctctgaaaacaat

SEQ ID NO: 33 - DNA, 20 bp, Artificial Sequence
  Synthetic - Primer
cagttccagggtctggtgtt

SEQ ID NO: 34 - DNA, 20 bp, Artificial Sequence
  Synthetic - Primer
gcaaagagtggtcggaagaa

SEQ ID NO: 35 - DNA, 24 bp, Artificial Sequence
  Synthetic - Primer
cgctctcgagtaaggtacatattg

SEQ ID NO: 36 - DNA, 20 bp, Artificial Sequence
  Synthetic - Primer
tggtctgggactctcttgat

SEQ ID NO: 37 - DNA, 20 bp, Artificial Sequence
  Synthetic - Primer
ggtcccaaacctgtcattct

SEQ ID NO: 38 - DNA, 20 bp, Artificial Sequence
  Synthetic - Primer
ctggaacggccaaatgtact

SEQ ID NO: 39 - DNA, 20 bp, Artificial Sequence
  Synthetic - Primer
gcttctttctccccctccta

SEQ ID NO: 40 - DNA, 20 bp, Artificial Sequence
  Synthetic - Primer
gcaacgtggtcatcacaaac

SEQ ID NO: 41 - DNA, 21 bp, Artificial Sequence
  Synthetic - Primer
ctcgatgaagcggtcatactc

SEQ ID NO: 42 - DNA, 22 bp, Artificial Sequence
  Synthetic - Primer
caatgtggatgccgcagttgtg

SEQ ID NO: 43 - DNA, 21 bp, Artificial Sequence
  Synthetic - Primer
cagcaccttggcggtctcgta

SEQ ID NO: 44 - DNA, 22 bp, Artificial Sequence
  Synthetic - Primer
agctctggaaagagggtttaag

SEQ ID NO: 45 - DNA, 21 bp, Artificial Sequence
  Synthetic - Primer
ctcctgtggcctgtcttattt

SEQ ID NO: 46 - DNA, 24 bp, Artificial Sequence
  Synthetic - Primer
tggttccaaaccagtttattctgt

SEQ ID NO: 47 - DNA, 24 bp, Artificial Sequence
  Synthetic - Primer
aagtgcgtttccatcatctttgag

SEQ ID NO: 48 - DNA, 22 bp, Artificial Sequence
  Synthetic - Primer
atgaccggaacaagaagaagaa

SEQ ID NO: 49 - DNA, 20 bp, Artificial Sequence
  Synthetic - Primer
ttgctgaccttggtgatgag

SEQ ID NO: 50 - DNA, 23 bp, Artificial Sequence
  Synthetic - Primer
cctggaggagaagaggaaagaga

SEQ ID NO: 51 - DNA, 25 bp, Artificial Sequence
  Synthetic - Primer
ttgaggacctctgtgtatttgtcaa

SEQ ID NO: 52 - DNA, 19 bp, Artificial Sequence
  Synthetic - Primer
gccaagggcgagtcccgta

SEQ ID NO: 53 - DNA, 27 bp, Artificial Sequence
  Synthetic - Primer
gcatcttgctcaactcggcgttgtgca

SEQ ID NO: 54 - DNA, 20 bp, Artificial Sequence
  Synthetic - Primer
gggtacgctgtagaccagac

SEQ ID NO: 55 - DNA, 20 bp, Artificial Sequence
  Synthetic - Primer
ttcagccgcttcacctgctt

SEQ ID NO: 56 - DNA, 20 bp, Artificial Sequence
  Synthetic - Primer
actcccaaagcagttggatg

SEQ ID NO: 57 - DNA, 21 bp, Artificial Sequence
  Synthetic - Primer
gcttttttatggcccaggctg

SEQ ID NO: 58 - DNA, 1221 bp, Artificial Sequence
  Synthetic - Human SOX17 cDNA
atgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatat